(19) United States
(12) Patent Application Publication
(10) Pub. No.: US 2020/0376029 A1
(43) Pub. Date: Dec. 3, 2020

(54) MEDICAMENT FOR CANCER TREATMENT

(71) Applicants: JSR Corporation, Tokyo (JP); KEIO University, Tokyo (JP)

(72) Inventors: Daiki Kato, Tokyo (JP); Tomonori Yaguchi, Tokyo (JP); Yutaka Kawakami, Tokyo (JP); Seiki Wakui, Tokyo (JP); Toru Shoji, Tokyo (JP); Ayuko Matsui, Tokyo (JP)

(21) Appl. No.: 15/931,789

(22) Filed: May 14, 2020

(30) Foreign Application Priority Data
May 15, 2019 (JP) 2019-092069

(51) Int. Cl. A61K 35/17; A61K 39/395; A61P 35/00

(52) U.S. Cl. CPC A61K 35/17; A61P 35/00; A61K 39/3955

(57) ABSTRACT

A medicament for cancer treatment includes as an active ingredient T cells having a chimeric antigen receptor that binds to glypican 1 (GPC1). The medicament is administered concomitantly with an immune checkpoint inhibitor according to regimens (a) and (b) to maintain the anti-tumor activity of the T cells: The regimens include (a) administering an effective amount of the T cells to a cancer patient and (b) continuously administering 0.01 mg/kg body weight to 100 mg/kg body weight of the immune checkpoint inhibitor per dose to the cancer patient every 1 to 5 weeks.

### TECHNICAL FIELD

The present invention relates to a medicament for cancer treatment. Priority is claimed on Japanese Patent Application No. 2019-092069 filed on May 15, 2019, the contents of which are incorporated herein by reference.

### BACKGROUND ART

Glypican 1 (GPC1) is a cell-surface heparan sulphate proteoglycan and is expressed in fetal tissues, tumor tissues, and the like. A major function of GPC1 is considered its involvement in the development of brain in fetal phase.

Knockout of GPC1 is known to result in no abnormalities morphology, behavior, or lifespan in adult mice, except for a slight decrease in brain volume in early fetal phase. GPC1 is considered to have no critical function in the body of a healthy adult. On the other hand, the overexpression of GPC1 has been reported in various tumors, including esophageal cancer, cervical cancer, breast cancer, pancreatic cancer, glioma, and mesothelioma. Further, Patent Literature 1 discloses a chimeric antigen receptor (hereinafter, sometimes referred to as “CAR”) specific to GPC1.

### CITATION LIST

### Patent Literature

### Patent Literature 1

PCT International Publication No. WO 2016/208754

### SUMMARY OF INVENTION

### Technical Problem

An object of the present invention is to provide a medicament composition, a kit, and a technique for effectively treating GPC1-positive cancer.

### Solution to Problem

The present invention includes the following aspects.

[1] A medicament for cancer treatment, including as an active ingredient T cells having a chimeric antigen receptor that binds to glypican 1 (GPC1), in which the medicament is administered concomitantly with an immune checkpoint inhibitor according to regimens (a) and (b) to maintain anti-tumor activity of the T cells:

(a) administering an effective amount of the T cells to a cancer patient; and

(b) continuously administering 0.01 mg/kg body weight to 100 mg/kg body weight of the immune checkpoint inhibitor per dose to the cancer patient every 1 to 5 weeks.

[2] The medicament for cancer treatment according to [1], in which the immune checkpoint inhibitor is at least one selected from the group consisting of an anti-PD-1 antibody, an anti-PD-L1 antibody, an anti-CTLA-4 antibody, an anti-TIGIT antibody, an anti-CD80 (B7-1) antibody, an anti-LAG-3 antibody, and an anti-TIM3 antibody.

[3] The medicament for cancer treatment according to [2], in which the immune checkpoint inhibitor is an anti-PD-1 antibody or an anti-TIGIT antibody.

[4] The medicament for cancer treatment according to [3], in which the immune checkpoint inhibitor is an anti-PD-1 antibody.

[5] The medicament for cancer treatment according to [4], in which the anti-PD-1 antibody is nivolumab.

[6] The medicament for cancer treatment according to any one of [1] to [5], in which the chimeric antigen receptor contains a GPC1 binding domain, a transmembrane domain, a costimulatory domain, and a cytoplasmic signal domain.

[7] The medicament for cancer treatment according to [6], in which the GPC1 binding domain includes a heavy chain variable region including a heavy chain CDR1 consisting of an amino acid sequence of SEQ ID NO: 9, a heavy chain CDR2 consisting of an amino acid sequence of SEQ ID NO: 10, and a heavy chain CDR3 consisting of an amino acid sequence of SEQ ID NO: 11, and a light chain variable region including a light chain CDR1 consisting of an amino acid sequence of SEQ ID NO: 12, a light chain CDR2 consisting of an amino acid sequence of SEQ ID NO: 13, and a light chain CDR3 consisting of an amino acid sequence of SEQ ID NO: 14.

[8] The medicament for cancer treatment according to [6] or [7], in which the GPC1 binding domain consists of a protein consisting of an amino acid sequence of SEQ ID NO: 15, or consists of a protein consisting of an amino acid sequence having a sequence identity of 95% or more with the amino acid sequence of SEQ ID NO: 15 and binds to GPC1.

[9] The medicament for cancer treatment according to any of [6] to [8], in which the GPC1 binding domain is humanized.

[10] The medicament for cancer treatment according to any one of [1] to [9], in which the cancer is a cancer selected from the group consisting of esophageal cancer, cervical cancer, breast cancer, pancreatic cancer, glioma, mesothelioma, thyroid cancer, lung cancer, liver cancer, colon cancer, head and neck cancer, urothelial cancer, ovarian cancer, melanoma, and prostate cancer.

[11] The medicament for cancer treatment according to [10], in which the cancer is esophageal cancer.

[12] An anti-tumor activity maintaining agent for T cells having a chimeric antigen receptor that binds to GPC1, the agent including as an active ingredient an immune checkpoint inhibitor, in which the agent is used by administering concomitantly with the T cells according to regimens (a) and (b):

(a) administering an effective amount of the T cells to a cancer patient; and

(b) continuously administering 0.01 mg/kg body weight to 100 mg/kg body weight of the immune checkpoint inhibitor per dose to the cancer patient every 1 to 5 weeks.

[13] A method for maintaining anti-tumor activity of T cells having a chimeric antigen receptor that binds to GPC1, the method including administering an immune checkpoint inhibitor concomitantly with the T cells to a patient in need thereof according to regimens (a) and (b):

(a) administering an effective amount of the T cells to a cancer patient; and

(b) continuously administering 0.01 mg/kg body weight to 100 mg/kg body weight of the immune checkpoint inhibitor per dose to the cancer patient every 1 to 5 weeks.

[14] A medicament for cancer treatment, including as an active ingredient a complex of a T cell having a chimeric antigen receptor that binds to GPC1 and an immune checkpoint inhibitor.

[15] The medicament for cancer treatment according to [14], in which the immune checkpoint inhibitor is at least one selected from the group consisting of an anti-PD-1 antibody, an anti-PD-L1 antibody, an anti-CTLA-4 antibody, an anti-TIGIT antibody, an anti-CD80 (B7-1) antibody, an anti-LAG-3 antibody, and an anti-TIM3 antibody.

[16] The medicament for cancer treatment according to [15], in which the immune checkpoint inhibitor is an anti-PD-1 antibody or an anti-TIGIT antibody.

[17] The medicament for cancer treatment according to [16], in which the immune checkpoint inhibitor is an anti-PD-1 antibody. [18] The medicament for cancer treatment according to [17], in which the anti-PD-1 antibody is nivolumab.

[19] A method for cancer treatment, including administering an effective amount of T cells having a chimeric antigen receptor that binds to GPC1 and an immune checkpoint inhibitor to a patient in need of treatment.

[20] The method for cancer treatment according to [19], in which the T cells and the immune checkpoint inhibitor are administered according to regimens (a) and (b):

(a) administering an effective amount of the T cells to a cancer patient; and

(b) continuously administering 0.01 mg/kg body weight to 100 mg/kg body weight of the immune checkpoint inhibitor per dose to the cancer patient every 1 to 5 weeks.

[21] The method for cancer treatment according to [19] or [20], in which the immune checkpoint inhibitor is an anti-PD-1 antibody.

[22] The method for cancer treatment according to [21], in which the anti-PD-1 antibody is nivolumab.

### Advantageous Effects of Invention

According to the present invention, it is possible to provide a medicament composition, a kit, and a technique for effectively treating GPC1-positive cancer.

### BRIEF DESCRIPTION OF DRAWINGS

FIG. 1A provides a graph showing the results of quantitative RT-PCR analysis in Experimental Example 1.

FIG. 1B provides representative micrographs of the results of immunostaining in Experimental Example 1.

FIG. 2A provides schematic diagrams showing the structures of hCAR generated in Experimental Example 2.

FIGS. 2B(a) to 2B(c) provide graphs showing the results of examining the expression of GPC1 in LK2-mock (hGPC1-negative lung cancer cell line), LK2-hGPC1 (hGPC1-overexpressing lung cancer cell line), and TE14 (hGPC1 endogenous esophageal cancer cell line) by flow cytometry analysis in Experimental Example 2, respectively.

FIG. 2C provides a graph showing the results of hIFNγ secretion assay in Experimental Example 2.

FIGS. 2D(a) to 2D(c) provide graphs showing the results of ⁵¹Cr release assay in Experimental Example 2.

FIG. 2E provides a graph showing the measurement results of tumor volume in Experimental Example 2.

FIG. 2F(a) provides a graph showing the results of examining the reactivity of anti-human TIGIT antibody (mouse, clone C18-25) against human TIGIT antigen.

FIG. 2F(b) provides a graph showing the evaluation results of cytotoxic activity of hCAR-T cells in combination with anti-human TIGIT antibody (mouse, clone C18-25) against LK2-hGPC1.

FIG. 3A provides a graph showing the results of quantitative RT-PCR analysis in Experimental Example 3.

FIG. 3B provides representative micrographs of the results of immunostaining in Experimental Example 3.

FIGS. 4A(a) to 4(d) provide graphs showing the results of examining the reactivity of anti-GPC1 monoclonal antibody (clone 1-12) or isotype control antibody by flow cytometry in Experimental Example 4.

FIG. 4B provides a schematic diagram showing the structure of mCAR generated in Experimental Example 4.

FIG. 4C provides a graph showing the results of IFNγ secretion assay in Experimental Example 4.

FIGS. 4D(a) and 4D(b) provide graphs showing the results of ⁵¹Cr release assay in Experimental Example 4.

FIGS. 4E(a) to 4E(c) provide graphs showing the measurement results of tumor volume of mice transplanted with MC38-mGPC1 cells in Experimental Example 4.

FIGS. 4F(a) to 4F(c) provide graphs showing the measurement results of tumor volume of mice transplanted with MCA205-mGPC1 cells in Experimental Example 4.

FIGS. 4G(a) and 4G(b) provide graphs showing the measurement results of ratio of GFP-positive CD8-positive mCAR-T cells in total CD8-positive T cells derived from peripheral blood (a) and tumor tissue (b), respectively, as measured in Experimental Example 4.

FIG. 4G(c) provides a graph showing the results of examination of long-term survival of mCAR-T cells by flow cytometry by detecting GFP-positive CD8-positive mCAR-T cells in peripheral blood on day 60 from the start of the experiment in Experimental Example 4.

FIGS. 4H(a) and 4H(b) provide graphs showing the measurement results of IFNγ concentration in Experimental Example 4.

FIG. 4I provides a graph showing the measurement results of mIFNγ concentration in Experimental Example 4.

FIGS. 5A(a) and 5A(b) provide graphs showing the results of measuring body weight in syngeneic mouse models on day 15 from the transplantation of cancer cells in Experimental Example 5.

FIG. 5B(a) provides micrographs showing the results of hematoxylin/eosin staining in Experimental Example 5.

FIG. 5B(b) provides micrographs showing the results of immunostaining in Experimental Example 5.

FIG. 6A(a) provides a graph showing the results of measuring PD-1 expression on endogenous CD8-positive T cells in Experimental Example 6.

FIG. 6A(b) provides a graph showing the results of measuring PD-1 expression on CD8-positive T cells administered in Experimental Example 6.

FIG. 6B provides a diagram showing the experimental schedule in Experimental Example 6.

FIG. 6C provides a graph showing the measurement results of tumor volume of mice in the respective groups in Experimental Example 6.

FIGS. 6D(a) to 6D(d) provide graphs showing the measurement results of tumor volume of mice in the respective groups in Experimental Example 6 for each individual mouse.

### DESCRIPTION OF EMBODIMENTS

[Notation of Gene and Protein Names]

In the present specification, human genes, mouse genes, and genes of other species are sometimes represented in uppercase letters without a strict distinction. Also, human proteins, mouse proteins, and proteins of other species are sometimes represented in uppercase letters without a strict distinction.

Unless otherwise specified, the components exemplarily described in the present specification may be used alone or in combination of two or more kinds. Further, when two or more kinds of components are used in combination, those components may be present as a single medicament composition.

[Kit for Cancer Treatment]

In one embodiment, the present invention provides a kit for cancer treatment including T cells having CAR that binds to GPC1 (CAR-T cell) and an immune checkpoint inhibitor. In this embodiment, CAR-T cells may be formed by the T cell itself expressing a CAR that binds to GPC1, or, for example, CAR-T cells may be formed by binding an NKG2D receptor of T cells expressing the NKG2D receptor to an anti-GPC1 antibody administered separately from the T cells. The anti-GPC1 antibody may be a fusion protein with the α1-α2 domain of the MHC-class 1-like Complex (MIC) protein, which is a ligand for the NKG2D receptor. Further, the NKG2D receptor may be a modified NKG2D receptor modified so as not to bind to the natural ligand, in which case the α1-α2 domain may be modified so as to bind to the modified NKG2D receptor described above. The anti-GPC1 antibody may be an antibody fragment such as scFV.

As will be described later in Examples, it was revealed that the use of CAR-T cell therapy targeting GPC1 in combination with anti-PD-1 antibody therapy shows a synergistic effect in cancer treatment. Therefore, according to the kit for cancer treatment of this embodiment, the GPC1-positive cancer can be effectively treated.

The NCBI accession number of mRNA of human GPC1 (hereinafter sometimes referred to as “hGPC1”) is NM_002081.2. The NCBI accession number of hGPC1 protein is NP_002072.2 or the like. The NCBI accession number of mRNA of mouse GPC1 (hereinafter sometimes referred to as “mGPC1”) is NM_016696.5 or the like. The NCBI accession number of mGPC1 protein is NP_057905.1 or the like.

In the kit for cancer treatment of this embodiment, the immune checkpoint inhibitor is preferably at least one selected from the group consisting of an anti-PD-1 antibody, an anti-PD-L1 antibody, an anti-CTLA-4 antibody, an anti-TIG1T antibody, an anti-CD80 (B7-1) antibody, an anti-LAG-3 antibody, and an anti-TIM3 antibody. The anti-4-1BB antibody is an agonist antibody that transmits a costimulatory signal that enhances T cell activity, but in the present specification, may be used in combination with an immune checkpoint inhibitor or instead of an immune checkpoint inhibitor.

Examples of the anti-PD-1 antibody include nivolumab and pembrolizumab. Examples of the anti-PD-L1 antibody include atezolizumab, avelumab, and durvalumab. Examples of the anti-CTLA-4 antibody include ipilimumab and tremelimumab. Examples of the anti-4-1BB antibody include utomilumab. The immune checkpoint inhibitors may be used alone or in combination of two or more kinds.

In the kit for cancer treatment of this embodiment, it is preferable that the CAR includes a GPC1 binding domain, a transmembrane domain, a costimulatory domain, and a cytoplasmic signal domain, and that the GPC1 binding domain binds to both hGPC1 and mGPC1.

Majority of preclinical studies on CAR transduced T cell (CAR-T cell) therapy have been conducted using xenogeneic mouse models in which human tumors are xenografted into immunodeficient mice. This approach is often the only option available for the development of CAR-T cell therapies targeting human tumors because CAR usually does not have cross-reactivity against homologous mouse antigens.

However, studies on CAR-T cell therapies in xenogeneic mouse models cannot adequately evaluate on-target/off-tumor toxicity. The on-target/off-tumor toxicity is considered mainly caused by CAR-T cells recognizing and attacking normal cells expressing the target antigen. In other words, off-tumor toxicity is a side effect. Since human normal cells (non-tumorous human cells) are absent in xenogeneic mouse models, the toxicity of CAR-T cells against them cannot be evaluated.

Here, if the CAR binds to both human target antigens and homologous mouse antigens (cross-reactive), the CAR-T cells can be tested using syngeneic mouse models in which mouse cancer is transplanted to healthy mice.

Since healthy mouse tissues express low levels of the target antigen, reflecting the pattern of expression in human patients, syngeneic mouse models can be used to elucidate the on-target/off-tumor toxicity of CAR-T cells.

As will be described later in Examples, the evaluation of CAR-T cells is interrupted in xenogeneic mouse models due to inappropriate host immunity and graft-versus-host disease (GVHD) by the xenografted T cells. On the other hand, the use of syngeneic mouse models would allow for a full evaluation of on-target/off-tumor toxicity as well as analyses of realistic factors that would significantly affect the efficacy of CAR-T cell therapies in humans, such as host anti-tumor immunity and interactions with species-specific immunosuppressive tumors.

In the kit for cancer treatment of this embodiment, it is preferable that the GPC1 binding domain of CAR includes a heavy chain variable region and a light chain variable region of anti-GPC1 antibody, and that the heavy chain variable region consists of the amino acid sequence of SEQ ID NO: 1, or consists of an amino acid sequence having a sequence identity of 95% or more with the amino acid sequence of SEQ ID NO: 1. Here, having a sequence identity of 95% or more means that the amino acid sequence is, for example, 95% or more, 96% or more, 97% or more, 98% or more, or 99% or more identical.

The GPC1 binding domain of CAR used in Examples described later is derived from an antibody generated by immunizing chickens. The GPC1 binding domain of CAR used in Examples has a heavy chain variable region having the amino acid sequence of SEQ ID NO: 1. Further, the GPC1 binding domain of CAR may have one or several mutation(s) in the heavy chain variable region having the amino acid sequence of SEQ ID NO: 1 as long as the GPC1 binding domain binds to GPC1.

In the present specification, the amino acid position assigned to the complementarity determining region (CDR) of the antibody is defined according to the definition by Kabat (Sequences of Proteins of Immunological Interest (Kabat E, Wu T, Perry H, Gottesman K.; National Institute of Health, Bethesda, Md., 1987 and 1991).

The light chain variable region of the GPC1 binding domain of CAR preferably consists of the amino acid sequence of SEQ ID NO: 2 or consists of an amino acid sequence having a sequence identity of 95% or more with the amino acid sequence of SEQ ID NO: 2. Here, having a sequence identity of 95% or more means that the amino acid sequence is, for example, 95% or more, 96% or more, 97% or more, 98% or more, or 99% or more identical.

The GPC1 binding domain of CAR used in Examples described later has a light chain variable region having the amino acid sequence of SEQ ID NO: 2. Further, the GPC1 binding domain of CAR may have one or several mutation(s) in the light chain variable region having the amino acid sequence of SEQ ID NO: 2 as long as the GPC1 binding domain binds to GPC1.

The GPC1 binding domain of CAR may be a single-chain antibody in which a heavy chain variable region, a linker, and a light chain variable region are linked in order from the N-terminus.

As will be described later in Examples, the present inventors have generated both a CAR in which the GPC1 binding domain is a single-chain antibody in which a heavy chain variable region, a linker, and a light chain variable region are linked in order from the N-terminus (hereinafter, sometimes referred to as “HL form”) and a CAR in which the GPC1 binding domain is a single-chain antibody in which a light chain variable region, a linker, and a heavy chain variable region are linked in order from the N-terminus (hereinafter, sometimes referred to as “LH form”), and their anti-tumor effects were examined. The results suggested that CAR-T cells having the CAR (HL form) in which the GPC1 binding domain is a single-chain antibody in which a heavy chain variable region, a linker, and a light chain variable region are linked in order from the N-terminus tend to show higher anti-tumor activity.

Although the linker for single-chain antibody may be any commonly used linker for single-chain antibody, it is preferable to use a linker consisting of the amino acid sequence of GGGGSGGGGSGGGGS (SEQ ID NO: 3), for example.

The heavy chain variable region of the GPC1 binding domain of CAR may consist of the amino acid sequence of SEQ ID NO: 1. Further, the light chain variable region of the GPC1 binding domain of CAR may consist of the amino acid sequence of SEQ ID NO: 2. The GPC1 binding domain of CAR may consist of the amino acid sequence of SEQ ID NO: 16 or may consist of an amino acid sequence having a sequence identity of 95% or more with the amino acid sequence of SEQ ID NO: 16, and the GPC1 binding domain of CAR may consist of the amino acid sequence of SEQ ID NO: 15 or may consist of an amino acid sequence having a sequence identity of 95% or more with the amino acid sequence of SEQ ID NO: 15, as long as the GPC1 binding domain binds to GPC1. Here, having a sequence identity of 95% or more means that the amino acid sequence is, for example, 95% or more, 96% or more, 97% or more, 98% or more, or 99% or more identical.

The heavy chain variable region and the light chain variable region of the GPC1 binding domain of CAR may be humanized. The expression “the heavy chain variable region and the light chain variable region are humanized” means that the framework region (FR) constituting the heavy chain variable region and the light chain variable region is substituted with an amino acid sequence derived from human or the like. The humanized antibody may be produced by constructing a cDNA encoding an amino acid sequence of a heavy chain variable region (VH) consisting of an amino acid sequence consisting of CDR of VH of a non-human antibody and an amino acid sequence of FR of VH of a human antibody and a cDNA encoding an amino acid sequence of a light chain variable region (VL) consisting of an amino acid sequence consisting of CDR of VL of a non-human antibody and an amino acid sequence of FR of VL of a human antibody, inserting these cDNAs into expression vectors having DNA encoding CH and CL of a human antibody respectively so as to construct an expression vector, and introducing the expression vector into cells for antibody expression. The humanized antibody may also be produced using an expression vector constructed by inserting a DNA encoding the heavy chain and a DNA encoding the light chain into a single expression vector.

When the GPC1 binding domain of CAR is humanized, side effects such as anaphylactic shock can be suppressed because the immunogenicity is low even when administered to human. This further allows CAR-T cells to be administered to a patient multiple times, for example.

Examples of cancers to be treated by the kit for cancer treatment of this embodiment include GPC1-positive cancers. In particular, examples thereof include esophageal cancer, cervical cancer, breast cancer, pancreatic cancer, glioma, mesothelioma, thyroid cancer, lung cancer, liver cancer, colon cancer, head and neck cancer, urothelial cancer, ovarian cancer, melanoma, and prostate cancer. The cancer to be treated by the kit for cancer treatment of this embodiment is preferably esophageal cancer. These cancers are known to express GPC1 and can be effectively treated by the kit for cancer treatment of this embodiment.

In the kit for cancer treatment of this embodiment, the T cells having CAR are preferably T cells whose rejection reaction is suppressed when transplanted into a patient. Specific examples of such T cells include patient-derived T cells, T cells whose HLA type matches the patient to an acceptable level, and T cells with suppressed expression of HLA protein. The T cells with suppressed expression of HLA protein may be genetically engineered cells. It may also be T cells generated by inducing differentiation from undifferentiated cells such as iPS cells or ES cells.

In the kit for cancer treatment of this embodiment, the CAR-T cells targeting GPC1 and the immune checkpoint inhibitor may be mixed and administered simultaneously, or may be administered separately. Alternatively, the CAR-T cells targeting GPC1 and the immune checkpoint inhibitor targeting GPC1 may be administered to a patient as a single medicament composition. Also, the immune checkpoint inhibitor may be administered multiple times after the administration of CAR-T cells targeting GPC1.

The CAR-T cells and the immune checkpoint inhibitor may each be formulated as separate medicament compositions, or may be formulated as a single medicament composition.

The medicament composition containing CAR-T cells may contain one or more kinds of pharmaceutically or physiologically acceptable carriers, additives, antibodies, and the like in addition to the CAR-T cells. Examples of the pharmaceutically or physiologically acceptable carrier include buffer solutions such as neutral buffered saline and phosphate buffered saline; carbohydrates such as glucose, mannose, sucrose, dextran, and mannitol; and amino acids such as glycine. Examples of the additive include cytokines such as IL-2, antioxidants, chelating agents such as EDTA, adjuvants such as aluminum hydroxide, and preservatives. Examples of the antibody include immune checkpoint inhibitors, and particularly preferable examples thereof include anti-PD-1 antibodies, anti-PD-L1 antibodies, anti-CTLA-4 antibodies, anti-TIGIT antibodies, anti-CD80 (B7-1) antibodies, anti-LAG-3 antibodies, and anti-TIM3 antibodies. The medicament composition containing CAR-T cells is preferably formulated for intravenous administration.

The medicament composition containing an immune checkpoint inhibitor may contain one or more kinds of pharmaceutically or physiologically acceptable carriers, additives, and the like.

Examples of the pharmaceutically or physiologically acceptable carrier include buffer solutions such as neutral buffered saline and phosphate buffered saline; carbohydrates such as glucose, mannose, sucrose, dextran, and mannitol; and amino acids such as glycine. Examples of the additives include antioxidants, chelating agents such as EDTA, adjuvants such as aluminum hydroxide, and preservatives. The medicament composition containing an immune checkpoint inhibitor is preferably formulated for intravenous administration.

The dose of CAR-T cells may be appropriately adjusted depending on factors such as the condition of the patient, the type and severity of the disease of the patient, but it is considered that the dose of about 1×10⁹ to 1×10¹² per patient is generally acceptable. The CAR-T cells may be administered only once or may be administered multiple times. In addition, chemotherapy may be administered prior to the administration of CAR-T cells. For example, cyclophosphamide (60 mg/kg body weight per day) and fludarabine (20 mg/m² (body surface area) per day) may be administered. Further, interleukin (IL)-2 may be administered from the day of administration at the time of administration of CAR-T cells. The administration regimen of IL-2 is exemplified by high-dose (720,000 IU/kg body weight up to 12 times every 8 hours) and low-dose (72,000 IU/kg body weight up to 15 times every 8 hours) in TIL therapy, and IL-2 may also be administered appropriately adjusting its dose in the CAR-T cell administration. (Steven A. Rosenberg., et al., A Phase I Study of Nonmyeloablative Chemotherapy and Adoptive Transfer of Autologous Tumor Antigen-Specific T Lymphocytes in Patients With Metastatic Melanoma, J Immunother. 2002; 25(3): 243-251.)

The dose of the immune checkpoint inhibitor may be appropriately adjusted depending on factors such as the condition of the patient, the type and severity of the disease of the patient. The initial administration may be concurrent with or separate from the administration of CAR-T cells. Tables 1 to 8 below show exemplary regimens of major treatments.

TABLE 1
Nivolumab PD-1

<Malignant melanoma>
Usually, for adults, nivolumab (genetic recombination) is given at a dose of 240 mg by
intravenous drip infusion every 2 weeks. In the case of adjuvant therapy for malignant
melanoma, the duration of administration is up to 12 months. When used in combination
with ipilimumab (genetic recombination) for unresectable malignant melanoma, usually, for
adults, nivolumab (genetic recombination) is given at a dose of 80 mg by intravenous drip
infusion 4 times every 3 weeks. Thereafter, nivolumab (genetic recombination) is given at
a dose of 240 mg by intravenous drip infusion every 2 weeks.
<Unresectable or metastatic renal cell carcinoma>
Usually, for adults, nivolumab (genetic recombination) is given at a dose of 240 mg by
intravenous drip infusion every 2 weeks. When used in combination with ipilimumab
(genetic recombination) for unresectable or metastatic renal cell carcinoma untreated with
chemotherapy, usually, for adults, nivolumab (genetic recombination) is given at a dose of
240 mg 4 times by intravenous drip infusion every 3 weeks. Thereafter, nivolumab
(genetic recombination) is given at a dose of 240 mg by intravenous drip infusion every 2
weeks.
<Unresectable advanced/recurrent non-small cell lung cancer, relapsed or refractory classical
Hodgkin lymphoma, recurrent or distant metastases head and neck cancer, curatively unresectable
advanced/recurrent gastric cancer exacerbated after cancer chemotherapy, unresectable
advanced/recurrent malignant pleural mesothelioma exacerbated after cancer chemotherapy,
curatively unresectable advanced/recurrent microsatellite instability-high (MSI-High) colorectal
cancer exacerbated after cancer chemotherapy, unresectable advanced/recurrent esophageal cancer
exacerbated after cancer chemotherapy>
Usually, for adults, nivolumab (genetic recombination) is given at a dose of 240 mg by
intravenous drip infusion every 2 weeks.

TABLE 2
Pembrolizumab PD-1

<Malignant melanoma>
Usually, for adults, pembrolizumab (genetic recombination) is given at a dose of 200
mg by intravenous drip infusion over 30 minutes every 3 weeks. In the case of
postoperative adjuvant therapy, the duration of administration is up to 12 months.
<Unresectable advanced/recurrent non-small cell lung cancer, relapsed or refractory classical
Hodgkin lymphoma, unresectable urothelial cancer exacerbated after cancer chemotherapy,
advanced/recurrent microsatellite instability-high (MSI-High) solid tumor exacerbated after
cancer chemotherapy (only when standard treatment is difficult), recurrent or distant
metastases head and neck cancer>
Usually, for adults, pembrolizumab (genetic recombination) is given at a dose of 200
mg by intravenous drip infusion over 30 minutes every 3 weeks.
<Unresectable or metastatic renal cell carcinoma>
In combined use with axitinib, usually, for adults, pembrolizumab (genetic recombination)
is given at a dose of 200 mg by intravenous drip infusion over 30
minutes every 3 weeks.

TABLE 3
Tecentriq PD-L1

<PD-L1-positive hormone receptor-negative and HER2-negative inoperable or recurrent breast cancer>
In combined use with paclitaxel (albumin suspension type), usually, for adults, atezolizumab
(genetic recombination) is given at a dose of 840 mg by intravenous drip infusion over 60
minutes every 2 weeks. If the initial administration is well tolerated, the administration
time of the second and subsequent administrations can be shortened to 30 minutes.
<Unresectable advanced/recurrent non-small cell lung cancer except squamous cell carcinoma
untreated with chemotherapy>
In combined use with other anti-neoplastic agents, usually, for adults, atezolizumab (genetic
recombination) is given at a dose of 1200 mg by intravenous drip infusion over 60 minutes
every 3 weeks. If the initial administration is well tolerated, the administration time of the
second and subsequent administrations can be shortened to 30 minutes.
<Unresectable advanced/recurrent non-small cell lung cancer treated with chemotherapy>
Usually, for adults, atezolizumab (genetic recombination) is given at a dose of 1200 mg by
intravenous drip infusion over 60 minutes every 3 weeks. If the initial administration is
well tolerated, the administration time of the second and subsequent administrations can be
shortened to 30 minutes.
<Progressive small cell lung cancer>
In combined use with carboplatin and etoposide, usually, for adults, atezolizumab (genetic
recombination) is given at a dose of 1200 mg by intravenous drip infusion over 60 minutes
every 3 weeks. If the initial administration is well tolerated, the administration time of the
second and subsequent administrations can be shortened to 30 minutes.

TABLE 4
Ipilimumab CTLA-4

<Unresectable malignant melanoma>
Usually, for adults, ipilimumab (genetic recombination)
is given at a dose of 3 mg/kg (body weight) by intravenous
drip infusion 4 times every 3 weeks. When used in
combination with other anti-neoplastic agents, nivolumab
(genetic recombination) needs to be used in combination.
<Unresectable or metastatic renal cell carcinoma>
In combined use with nivolumab (genetic recombination),
usually, for adults, ipilimumab (genetic recombination)
is given at a dose of 1 mg/kg (body weight) by
intravenous drip infusion 4 times every 3 weeks.

TABLE 5
Opdivo PD-1

<Unresectable or metastatic melanoma>
240 mg every 2 weeks or 480 mg every 4 weeks.
1 mg/kg followed by ipilimumab 3 mg/kg on the same day every 3 weeks for 4 doses,
then 240 mg every 2 weeks or 480 mg every 4 weeks.
<Adjuvant treatment of melanoma>
240 mg every 2 weeks or 480 mg every 4 weeks.
<Melaslatic non-small cell lung cancer>
240 mg every 2 weeks or 480 mg every 4 weeks.
<Small cell lung cancer>
240 mg every 2 weeks.
<Advanced renal cell carcinoma>
240 mg every 2 weeks or 480 mg every 4 weeks.
3 mg/kg followed by ipilimumab 1 mg/kg on the same day every 3 weeks for 4 doses,
then 240 mg every 2 weeks or 480 mg every 4 weeks.
<Classical Hodgkin lymphoma>
240 mg every 2 weeks or 480 mg every 4 weeks.
<Recurrent or metastatic squamous cell carcinoma of the head and neck>
240 mg every 2 weeks or 480 mg every 4 weeks.
<Locally advanced or metastatic urothelial carcinoma>
240 mg every 2 weeks or 480 mg every 4 weeks.
<Microsatcllite instability-high (MSI-H)
or mismatch repair deficient (dMMR)
metastatic colorectal cancer>
Adult and pediatric patients ≥40 kg: 240 mg every 2 weeks or 480 mg every 4 weeks.
Pediatric patients <40 kg: 3 mg/kg every 2 weeks.
Adult and pediatric patients ≥40 kg: 3 mg/kg followed by ipilimumab 1 mg/kg on the
same day every 3 weeks for 4 doses, then 240 mg every 2 weeks or 480 mg every 4 weeks.
<Hepatocellular carcinoma>
240 mg every 2 weeks or 480 mg every 4 weeks.
1 mg/kg followed by ipilimumab 3 mg/kg on the same day every 3 weeks for 4 doses,
then 240 mg every 2 weeks or 480 mg every 4 weeks.

TABLE 6
Pembrolizumab PD-1

<Melanoma>
200 mg every 3 weeks.
<NSCLC>
200 mg every 3 weeks.
<HNSCC>
200 mg every 3 weeks.
<cHL or PMBCL>
200 mg every 3 weeks for adults;
2 mg/kg (up to 200 mg)
every 3 weeks for pediatrics.
<Urothelial Carcinoma>
200 mg every 3 weeks.
<MSI-H Cancer>
200 mg every 3 weeks for adults and
2 mg/kg (up to 200 mg)
every 3 weeks for pediatrics.
<Gastric Cancer>
200 mg every 3 weeks.
<Cervical Cancer>
200 mg every 3 weeks.
<HCC>
200 mg every 3 weeks.
<MCC>
200 mg every 3 weeks for adults;
2 mg/kg (up to 200 mg)
every 3 weeks for pediatrics.
<RCC>
200 mg every 3 weeks with axitinib 5 mg orally twice daily.

TABLE 7
Tecentriq PD-L1

<Urothelial Carcinoma>
840 mg TV q2 Weeks or 1200 mg IV q3 Weeks or 1680 mg IV q4 Weeks
<Non-Small Cell Lung Cancer>
Single agent for disease progression 840 mg IV q2 Weeks or 1200 mg IV q3 Weeks or 1680 mg IV q4 Weeks
Combination therapy with bevacizumab, paclitaxel, and carboplatin
Indicated in combination with bevacizumab, paclitaxel, and carboplatin for first-line treatment
of patients with metastatic nonsquamous NSCLC with no EGFR or ALK genomic tumor aberrations OR
1200 mg IV on Day 1 q3 Weeks plus bevacizumab, paclitaxel, and carboplatin x4-6 cycles
Refer to prescribing information for bevacizumab, paclitaxel, and carboplatin for recommended dosing
information
After completion of chemotherapy cycles 4-6 with bevacizumab Atezolizumab 1200 mg IV, followed by
bevacizumab on Day 1 q3 Weeks; continue until disease progression or unacceptable toxicity
Atezolizumab dose following completion of 4-6 cycles, and if bevacizumab is discontinued
840 mg IV q2 Weeks or
1200 mg IV q3 Weeks or
1680 mg IV q4 Weeks
Continue until disease progression or unacceptable toxicity
Combination therapy with paclitaxel protein-bound and carboplatin
Indicated in combination with paclitaxel protein-bound and carboplatin for first-line treatment
of patients with metastatic nonsquamous NSCLC with no EGFR or ALK genomic tumor aberrations
Atezolizumab 1200 mg on day 1 q3 Weeks plus paclitaxel protein-bound
and carboplatin x 4-6 cycles for each 21-day cycle
Refer to prescribing information for paclitaxel protein-bound and
carboplatin for recommended dosing information
Atezolizumab dose following completion of 4-6 cycles
840 mg IV q2 Weeks or
1200 mg IV q3 Weeks or
1680 mg IV q4 Weeks
Continue until disease progression or unacceptable toxicity
After completion of chemotherapy cycles 4-6 with bevacizumab
Atezolizumab 1200 mg IV, followed by bevacizumab on Day 1 q3 Weeks;
continue until disease progression or unacceptable toxicity
Atezolizumab dose following completion of 4-6 cycles
840 mg IV q2 Weeks or
1200 mg IV q3 Weeks or
1680 mg IV q4 Weeks
Continue until disease progression or unacceptable toxicity
<Triple-Negative Breast Cancer>
Atezolizumab 840 mg IV on Days 1 and 15 for each 28-day cycle PLUS
Paclitaxel protein-bound 100 mg/m² on Days 1, 8, and 15 for each 28-day cycle
Continue until disease progression or unacceptable toxicity
<Small Cell Lung Cancer>
Following completion of 4 cycles of carboplatin and etoposide
840 mg IV q2 Weeks or
1200 mg IV q3 Weeks or
1680 mg IV q4 Weeks
Continue until disease progression or unacceptable toxicity

TABLE 8
Yervoy CTLA-4

<Malignant Melanoma>
Unresectable or metastatic melanoma
3 mg/kg IV q3 Week for a maximum of 4 doses
Adjuvant treatment
10 mg/kg IV q3 Week for 4 doses followed by 10 mg/kg q12 Week for up to 3 yr
<Renal Cell Carcinoma>
1 mg/kg IV q3 Weeks following nivolumab on the same day;
repeat for up to 4 doses or until intolerable toxicity or disease progression
<Microsatellite Instability-High or Mismatch Repair Deficient Metastatic Colorectal Cancer>
1 mg/kg IV q3 Weeks following nivolumab on the same day;
repeat for up to 4 doses or until intolerable toxicity or disease progression
<Hepatocellular Carcinoma>
3 mg/kg IV q3 Weeks following nivolumab on the same day;
repeat for up to 4 doses or until intolerable toxicity or disease progression

### OTHER EMBODIMENTS

In one embodiment, the present invention provides a method for cancer treatment, including administering an effective amount of T cells having CAR that binds to GPC1 and an immune checkpoint inhibitor to a patient in need thereof. In this embodiment, CAR that binds to GPC1, T cells having CAR, immune checkpoint inhibitor, dose, administration method and the like are as described above.

### EXAMPLES

Next, the present invention will be described in more detail with reference to examples, but the present invention is not limited to the following examples.

[Materials and Methods]

(Tissue Sample)

Frozen tissue array slides of normal human tissues (FDA standard frozen tissue array-human adult normal, T6234701-1) were purchased from Biochain. Frozen tissue slides of normal mice (C57BL/6) were self-made. In order to evaluate side effects, mice transplanted with T cells were fixed by perfusion with 4% paraformaldehyde, then systemic tissues were collected, and the collected tissues were fixed with formalin and embedded in paraffin.

(Quantitative RT-PCR Analysis)

Preparation of total RNA, cDNA synthesis, and real-time RT-PCR were performed using standard protocols. The GAPDH gene was used as a housekeeping gene for quantitative real-time PCR normalization. TaqMan probe (catalog number “Mm00497305_m1”) and RT-PCR primer (GPC1) for quantification of hGPC1 gene and mGPC1 gene were purchased from Thermo Fisher Scientific.

(Immunostaining)

Immunostaining was performed using standard protocols on frozen sections (for GPC1) or formalin-fixed, paraffin-embedded sections (for GFP) of human and mouse systemic tissues. An anti-GPC1 antibody (clone 1-12, 0.5 μg/mL, Harada E., et al., Glypican-1 targeted antibody-based therapy induces preclinical antitumor activity against esophageal squamous cell carcinoma, Oncotarget, 8(15), 24741-24752, 2017) and an anti-GFP antibody (clone 1E4, 0.5 μg/mL, M048-3, Medical & Biological Laboratories Co., Ltd.) were used as primary antibodies.

(Cell Line and Medium)

A human esophageal squamous cell carcinoma cell line (TE14), human lung squamous cell carcinoma cell line LK2 in which hGPC1 was forcibly expressed (LK2-hGPC1), and a cell line in which an empty vector is introduced into LK2 (LK2-mock) were used. A murine colon adenocarcinoma cell line (MC38), a murine sarcoma cell line (MCA-205), and a murine T cell lymphoma (EL4) were obtained from National Cancer Institute (USA).

In addition, MC38 and MCA205 cells were also transduced with a lentiviral vector encoding mGPC1 cDNA. Hereinafter, MC38 and MCA205 that stably express mGPC1 are sometimes referred to as MC38-mGPC1 and MCA205-mGPC1, respectively.

An AIM-V medium (Thermo Fisher Scientific, DK 0870112) containing 10% heat-inactivated human AB serum (GEMINI, 100-512) and 300 IU/mL recombinant human interleukin-2 (rhIL-2, NIPRO, 87-890) was used as the medium for human T cells. An RPMI medium containing 10% heat-inactivated fetal bovine serum (FBS, SIGMA, 172012-500 ml), 100 U/mL penicillin, 100 μg/mL streptomycin, 0.05 mM 2-mercaptoethanol, 0.1 mM MEM non-essential amino acids, 1 mM sodium pyruvate, 10 mM HEPES (Gibco, 15630-080), and 50 IU/mL genetic recombinant human IL-2 (rhIL-2) (Nacalai Tesque, 30264-56) was used as the medium for murine T cells.

(Retroviral Vector Design)

The nucleotide sequence encoding an anti-GPC1 single chain variable region fragment (scFv) in VL-VH (LH form) or VH-VL (HL form) format was generated based on the nucleotide sequence of an anti-GPC1 antibody (Clone 1-12) recognizing human and mouse GPC1.

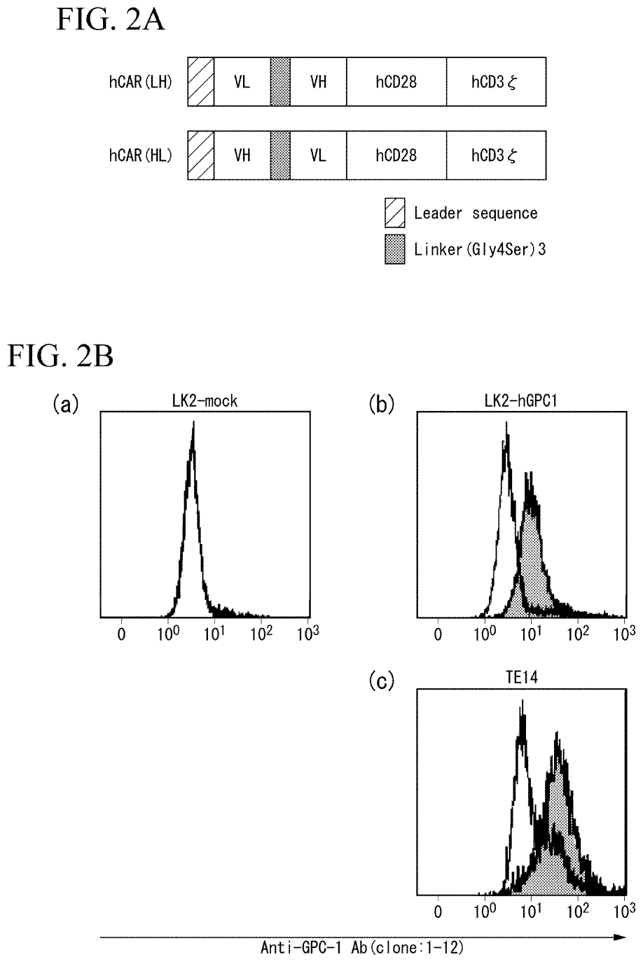

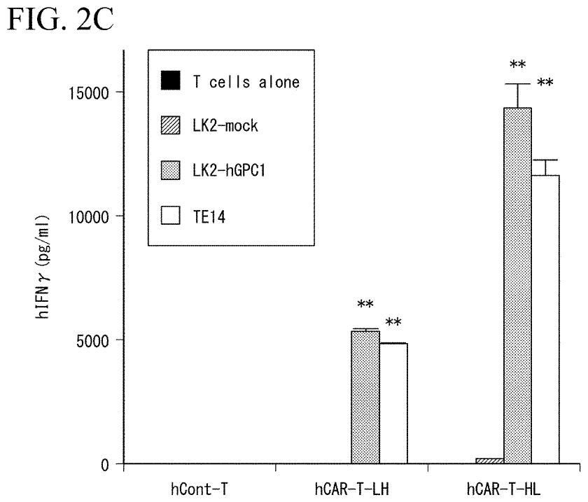

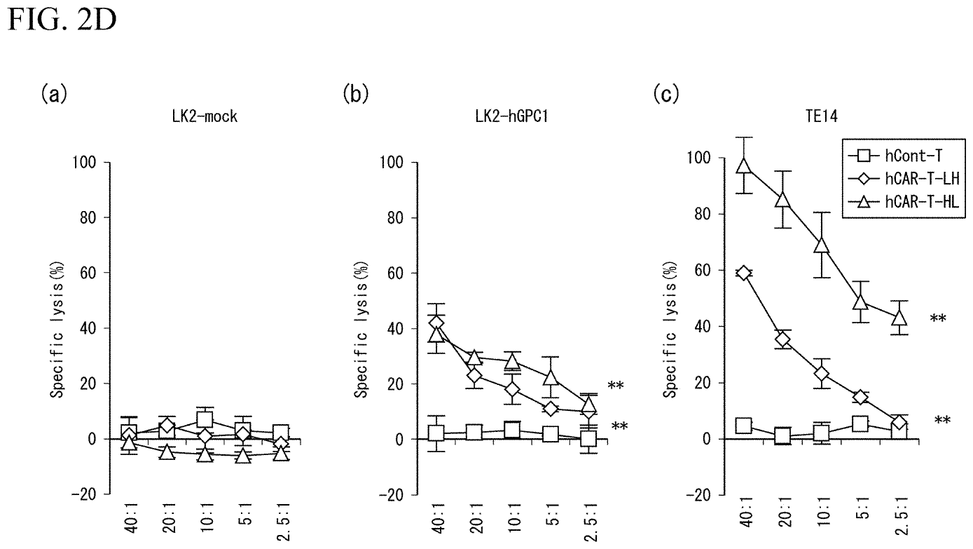

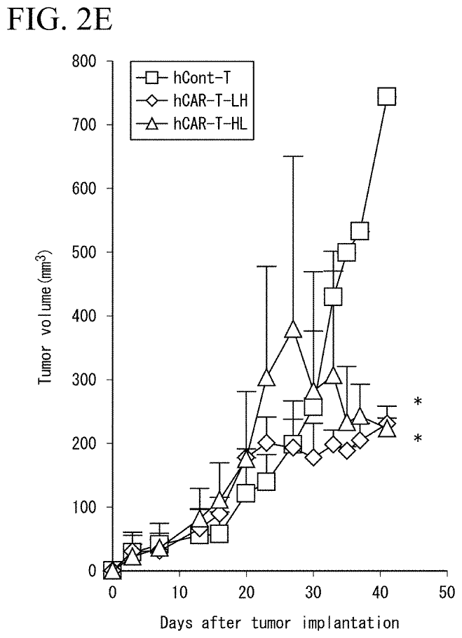

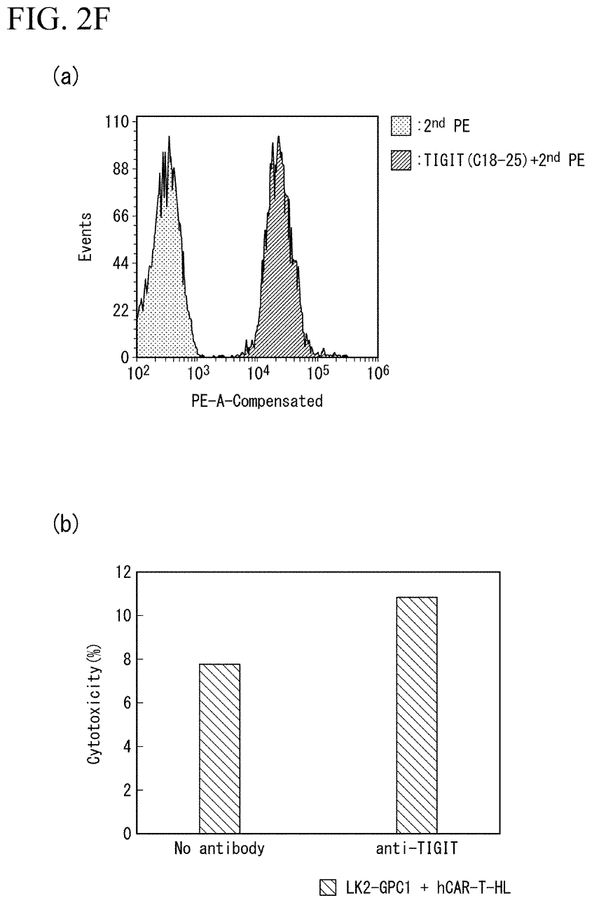

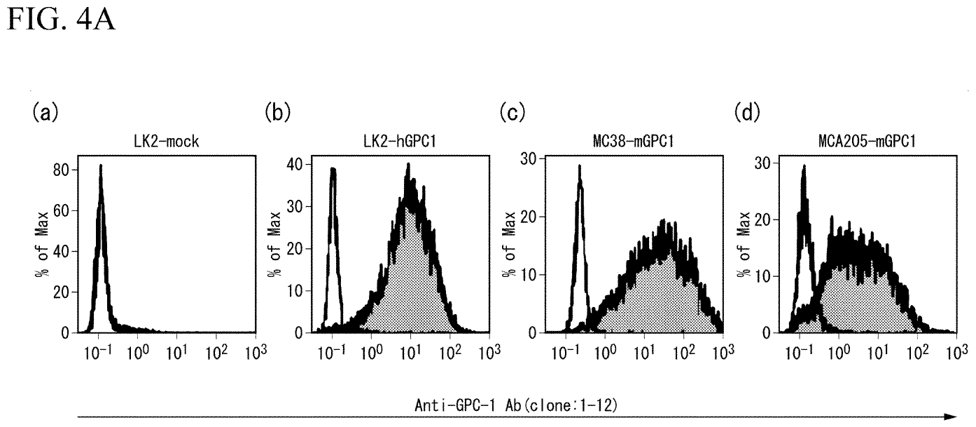

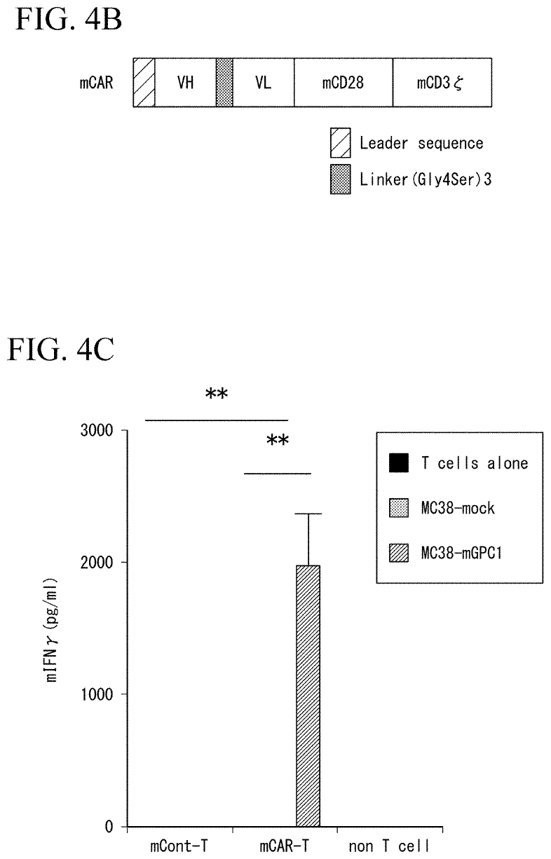

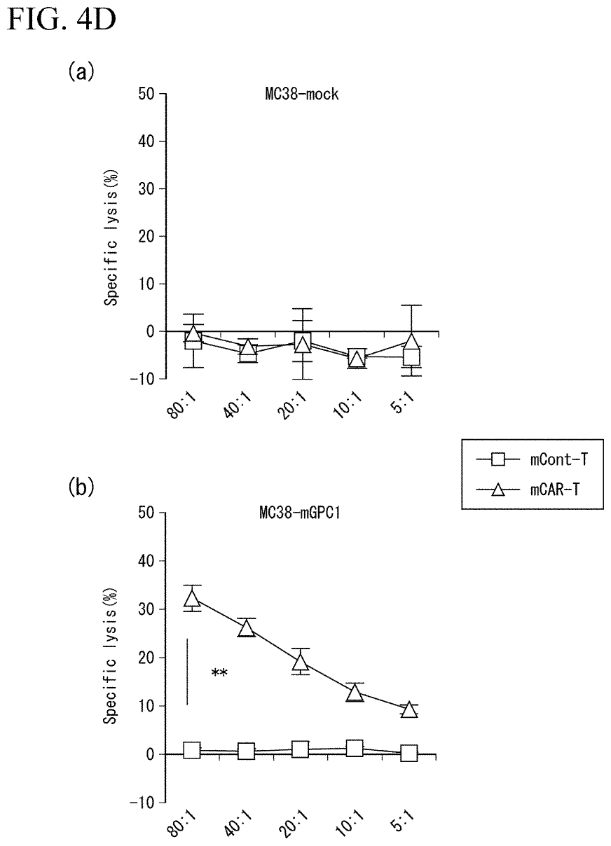

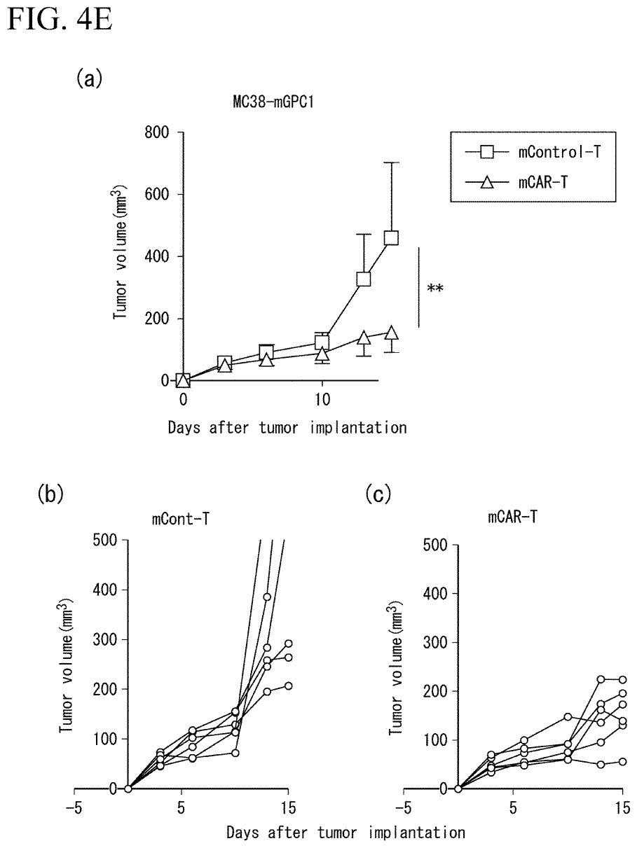

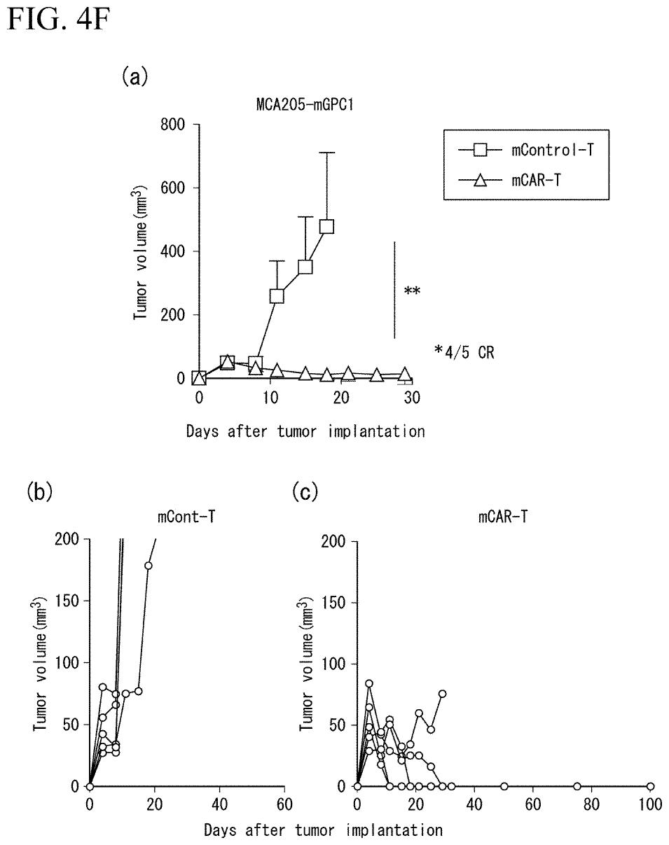

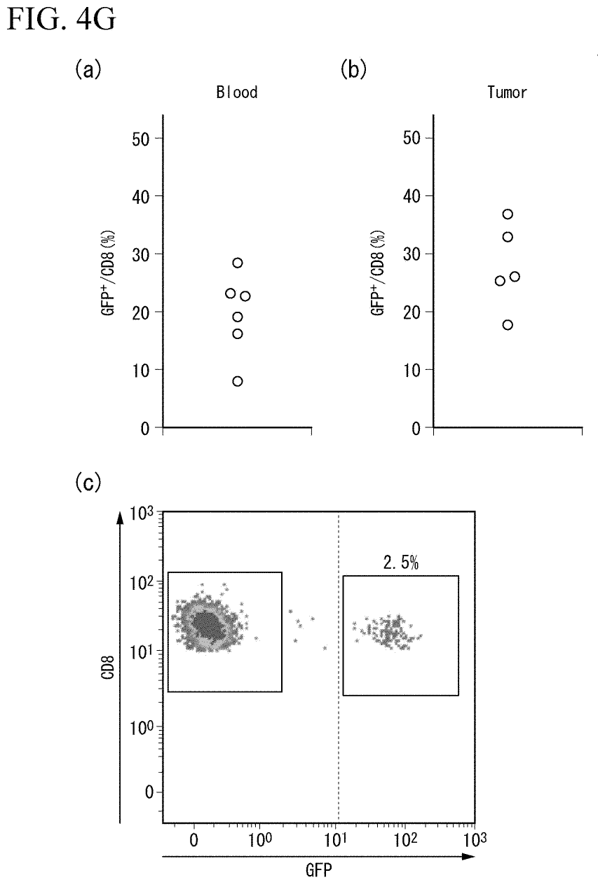

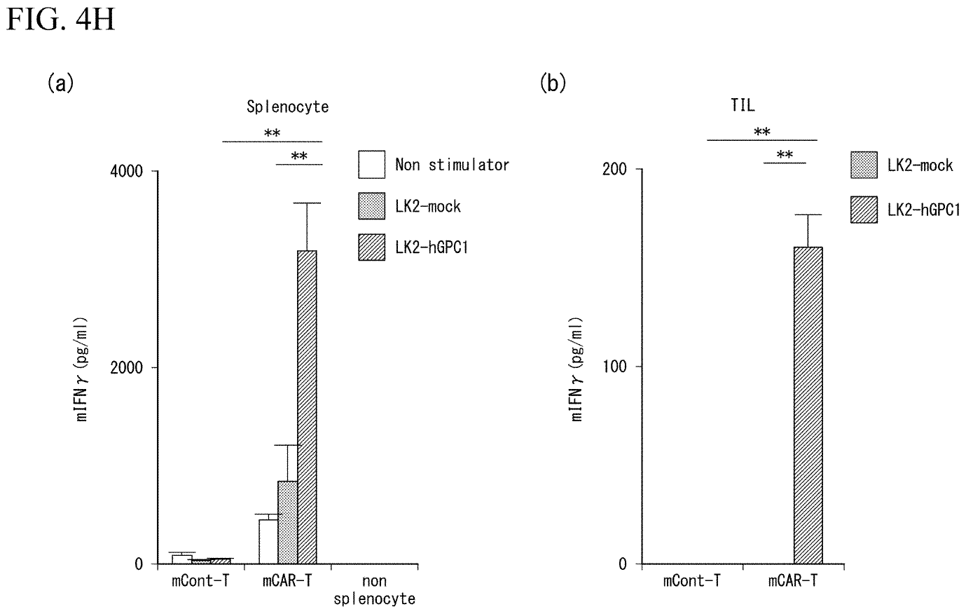

As shown in FIG. 2A, human CAR (hCAR) contains the human CD8a leader sequence, anti-GPC1 scFv, human CD28 extracellular domain/transmembrane domain/intracellular domain, and human CD3S intracellular domain. The nucleotide sequence of hCAR (LH form) is shown in SEQ ID NO: 4. The nucleotide sequence of hCAR (HL form) is shown in SEQ ID NO: 5. As shown in FIG. 4A, mouse CAR (mCAR) contains the mouse CD8a leader sequence, anti-GPC1 scFv, mouse CD28 extracellular domain/transmembrane domain/intracellular domain, and mouse CD3 intracellular domain. The nucleotide sequence of mCAR is shown in SEQ ID NO: 6. These hCAR and mCAR genes were cloned in-frame into a retroviral vector.

(Preparation of hCAR-T Cells and mCAR-T Cells)

The GPC1-specific hCAR gene (LH form or HL form) or mCAR gene (HL form) was introduced into stimulated human T cells or murine T cells. First, transient retroviral supernatants were generated by co-transfecting into G3Thi cells with a human or mouse CAR plasmid, a plasmid encoding an ECO envelope, and a plasmid encoding gag-pol, using Hily Max (Dojindo Laboratories, H357). After 12 hours, the supernatants were replaced with a fresh medium, and the retroviral supernatants were harvested 24 hours after replacement of the medium. This retrovirus was used for the generation of mCAR-T cells. For the generation of hCAR-T cells, the retrovirus was further infected with PG13 cells and a GaLV envelope retrovirus produced in the supernatant was used. The respective retroviral supernatants were centrifuged at 2000×g at 32° C. for 2 hours on a plate coated with RetroNectin ((registered trademark) TAKARA BIO INC., T100B) to prepare a retroviral plate.

For the generation of hCAR-T cells, peripheral blood mononuclear cells from healthy donors were subsequently stimulated with soluble OKT-3 (50 ng/mL, BioLegend, 317347) for 2 days before transduction. Subsequently, the stimulated cells were seeded on a retroviral plate and transduced by spinning down at 1000×g for 10 minutes.

For the generation of mCAR-T cells, mouse splenocytes were harvested from a transgenic mouse constitutively expressing EGFP under the control of the CAG promoter, and activated with 2.5 mg/mL concanavalin A (Sigma Aldrich, Inc., C5275) on day 0. Subsequently, 1 day prior to transduction, mouse splenocytes were stimulated with 2 μg/mL concanavalin A, 1 ng/mL recombinant mouse interleukin 7 (rmIL-7, Papertec, 217-17) and 50 IU/mL human IL-2. Subsequently, the stimulated splenocytes were transduced by spinning down on a retroviral plate at 1000×g for 10 minutes.

(Flow Cytometry Analysis)

Tumor cell lines were stained with anti-GPC1 antibody (clone 1-12). Regarding the transduced T cells, CAR expressed on the cell surface was stained with an APC-labeled donkey anti-chicken IgY antibody (Jackson, 703-136-155). The anti-GPC1 antibody (clone 1-12) was an antibody derived from chicken. Mouse cells in tissue and blood were stained with anti-CD45 antibody (V500, 30-F11, BD Biosciences, 561487), anti-CD3 antibody (BV-421, 145-2C11, BioLegend, 100336) and anti-CD8 antibody (Alexa Fluor 700, 53-6.7, BD Biosciences, 557959).

(Flow Cytometry Analysis, Evaluation of Anti-TIGIT Antibody)

TIGIT expressed on the cell surface of HEK293 TIGIT (hTIGIT-overexpressing human fetal renal cell line) was stained using anti-human TIGIT antibody (mouse) (Medical & Biological Laboratories, clone C18-25) and as a secondary antibody PE-labeled anti-mouse immunoglobulin antibody (Poly 4053, BioLegend, 405307).

(Cytotoxic Activity)

LK2-hGPC1 was labeled with carboxyfluorescein succinimidyl ester (CFSE, Dojindo Laboratories, C375), anti-human TIGIT antibody was added to a volume of 10 μg/mL, and the same number of GPC1-specific CAR-T cells as LK2-hGPC1 were added. After incubation at 37° C. for 6 hours, the cells were redispersed in Annexin-V-Binding Buffer (BioLegend, 422201) and then APC Annexin V (BioLegend, 640941) was added, and apoptotic cells and necrotic cells were evaluated by flow cytometry.

(IFNγ Secretion Assay)

Cultured tumor cell lines were used as stimulator cells. T cells were co-cultured with stimulator cells and the supernatant was harvested 24 hours later. Human and mouse interferon (IFN) γ concentrations were measured using a human IFNγ (hIFNy) ELISA set (Endogen: M700A and M700B) or a mouse IFNγ (mIFNy) ELISA set (BD Biosciences, 555138), respectively.

(Chromium Release Assay)

Target cells were labeled with ⁵¹Cr and mixed with transduced cells in multiple mixing ratios. After incubating at 37° C. for 4 hours, the release of free ⁵¹Cr was measured (Yaguchi T., et al., Immune Suppression and Resistance Mediated by Constitutive Activation of Wnt/β-Catenin Signaling in Human Melanoma Cells, J Immunol, 189 (5), 2110-2117, 2012).

(Mouse Model Analysis)

In xenogeneic mouse models, 3×10⁶ TE14 cells were subcutaneously inoculated into the flank of 6-10 week-old (NOD-scid IL-2γ null, NOG) mice. Cultured hCAR-T cells or hCont-T cells (2×10⁷ cells/mouse) were intravenously administered on day 9. Here, hCont-T cells mean human T cells not transduced with CAR.

In syngeneic mouse models, 5×10⁵ MC38-mGPC1 or MCA205-mGPC1 cells were subcutaneously inoculated into the flank of 6-8 week old C57BL/6 mice. Cultured mCAR-T cells or mCont-T cells (2×10⁶ cells/mouse) were intravenously administered on day 3. Here, mCont-T cells mean murine T cells not transduced with CAR. Immediately before T cell transplantation, the mice were irradiated with 5 Gy of systemic radiation, and rhIL-2 was intraperitoneally administered twice a day. The rhIL-2 was administered 6 times at a dose of 50,000 IU/mouse.

In combination therapy models, 200 mg/mouse of anti-PD-1 antibody (J43, Bio X Cell, BE0033-2) or isotype antibody (PIP, Bio X Cell, BE0260) was injected intraperitoneally on days 2, 6, 14, and 18. The tumor volume was calculated according to the following formula (1).

Tumor volume=[(length)×(width)]/2 (1)

(Evaluation of Tumor-Specific CD8-Positive T Cell Response)

In order to evaluate the immune response of CD8-positive tumor-infiltrating T cells specific for endogenous tumor antigens, sorted CD8-positive T cells were restimulated with EL4 cells pulsed with 1 mg/mL gp70 peptide (SEQ ID NO: 7) or control peptide (peptide derived from H-2Kb-restricted β-Galactosidase (β-gal), SEQ ID NO: 8) for 24 hours and the IFNγ secretion was evaluated.

(Statistical Analysis)

All results are expressed as mean value ±standard deviation. Statistical analysis of the data (unpaired t test and Bonferroni/Dunn's test) was performed to determine the differences between the means of the experimental, treated, and control groups. GraphPad Prism 7.0 was used for statistical calculation. P<0.05 (*) and P<0.01 (**) were considered to be statistically significant.

### Experimental Example 1

(Examination on Expression of hGPC1 in Human Adult Normal Systemic Tissues)

The expression of hGPC1 in human adult normal systemic tissues was examined First, in order to evaluate the specificity of hGPC1 expression at mRNA level, the expression level of hGPC1 mRNA in fetal brain tissue was compared to the expression level of hGPC1 mRNA in various adult human normal tissues by quantitative RT-PCR analysis. GAPDH mRNA was used as an internal control.

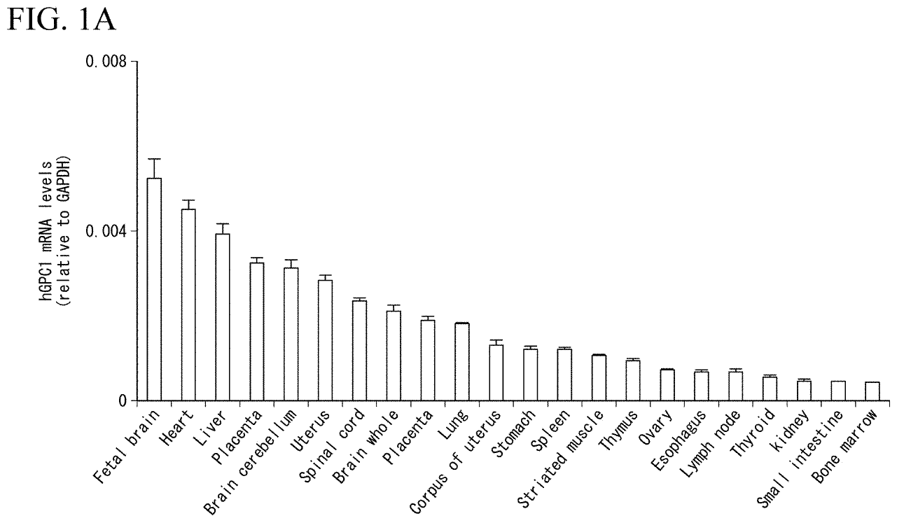

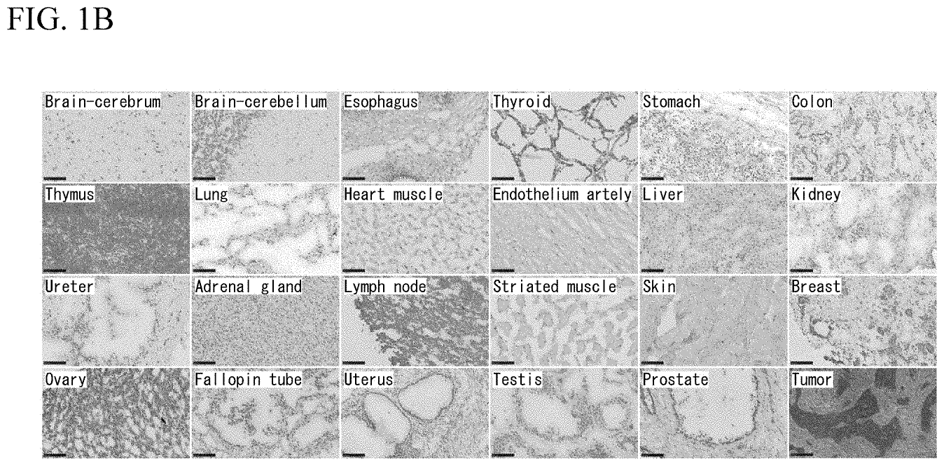

FIG. 1A provides a graph showing the results of quantitative RT-PCR analysis. As a result, it was revealed that a slight, but detectable, expression of hGPC1 mRNA was observed in almost all tissues.

Subsequently, the reactivity of anti-GPC1 monoclonal antibody (clone 1-12) that specifically reacts with hGPC1 and mGPC1 was evaluated against various human normal tissues.

FIG. 1B provides representative micrographs of the results of immunostaining. The scale bar indicates 100 μm. As a result, it was revealed that normal tissues are not clearly stained by anti-GPC1 antibody (clone 1-12), in contrast to the strong expression of GPC1 in human esophageal cancer tissues. Similar results were confirmed for tissue samples from three donors of different ages and genders.

From the above results, it was revealed that although hGPC1 mRNA is detected by quantitative RT-PCR, the expression of hGPC1 at protein level is not detected in human adult normal tissues. This result indicates that GPC1 is a promising therapeutic target for CAR-T cell therapies and that anti-GPC1 monoclonal antibody (clone 1-12) can be used for the development of GPC1-specific CAR-T cell therapy.

### Experimental Example 2

(Examination of GPC1-Specific hCAR-T Cells)

A retrovirus expression vector encoding a GPC1 specific hCAR (hCAR, LH form, and HL form) containing a scFv fragment containing a light chain and a heavy chain derived from anti-GPC1 monoclonal antibody (clone 1-12), and containing signal domains of human CD28 and CD3ζ was generated.

FIG. 2A provides schematic diagrams showing the structures of hCAR. As shown in FIG. 2A, the LH form has a light chain variable region (VL) and a heavy chain variable region (VH) in order from the N-terminus. Further, the HL form has a heavy chain variable region (VH) and a light chain variable region (VL) from the N-terminus in order.

Subsequently, the generated hCAR vector was transduced into human activated T cells. In both LH and HL forms, the transduction efficiency of transduced T cells (hCAR-T cells) was about 60%. There was no significant difference between the LH form and the HL form in the proliferation ability of hCAR-T cells after transduction.

IFNγ secretion and tumor cell (target cell) killing activity by T cells in response to target antigens are important in antigen-specific anti-tumor immune responses. The present inventors tested these abilities of the CAR-T cells by IFNγ secretion assay and ⁵¹Cr release assay.

FIGS. 2B(a) to 2B(c) provide graphs showing the results of examining the expression of GPC1 in LK2-mock (hGPC1-negative lung cancer cell line), LK2-hGPC1 (hGPC1-overexpressing lung cancer cell line), and TE14 (hGPC1 endogenous esophageal cancer cell line) by flow cytometry analysis, respectively. hCAR-T cells were co-cultured with these cells and subjected to IFNγ secretion assay and ⁵¹Cr release assay.

FIG. 2C provides a graph showing the results of IFNγ secretion assay. In FIG. 2C, “hCont-T” indicates the result of human T cells not transduced with CAR, “hCAR-T-LH” indicates the result of T cells transduced with LH form hCAR, “hCAR-T-HL” indicates the result of T cells transduced with HL form hCAR, “T cells alone” indicates the result of culturing only various T cells for comparison, “LK2-hGPC1” indicates the result of co-culturing various cells with LK2-hGPC1 cells, and “LK2-mock” indicates the result of co-culturing various T cells with LK2-mock cells, and “TE14” indicates the result of co-culturing various T cells with TE14 cells. As a result, it was revealed that hCAR-T cells released IFNγ in a hGPC1-specific manner. When CAR-T cells recognize an antigen, they produce IFNγ and exert a cytotoxic activity, and therefore increased production of IFNγ indicates that hCAR-T cells target LK2-hGPC1.

FIGS. 2D(a) to 2D(c) provide graphs showing the results of the ⁵¹Cr release assay. In FIGS. 2D(a) to 2D(c), “hCont-T” indicates the result of human T cells not transduced with CAR, “hCAR-T-LH” indicates the result of T cells transduced with LH form hCAR, and “hCAR-T-HL” indicates the result of T cells transduced with HL form hCAR. In FIGS. 2D(a) to 2D(c), the vertical axis represents the specific lysis of cells measured by the release of ⁵¹Cr, and the horizontal axis represents the mixing ratio of T cells and cancer cells (T cells: cancer cells).

FIG. 2D(a) shows the result of co-culturing various T cells with LK2-mock cells, FIG. 2D(b) shows the result of co-culturing various T cells with LK2-hGPC1 cells, and FIG. 2D(c) shows the result of co-culturing various T cells with TE14 cells. As a result, it was revealed that hCAR-T cells released IFNγ in a hGPC1-specific manner and killed the target cells.

Subsequently, the present inventors intravenously administered the hCAR-T cells to TE14 xenografted NOG mice, and examined anti-tumor activities of the CAR-T cells in vivo. hCAR-T cells (LH form and HL form) or human T cells not transduced with CAR were intravenously administered at a dose of 2×10⁷ cells/mouse, and the time course of tumor volume was measured.

FIG. 2E provides a graph showing the measurement results of tumor volume. In FIG. 2E, “hCont-T” indicates the result of mice administered with human T cells not transduced with CAR, “hCAR-T-LH” indicates the result of mice administered with T cells transduced with LH form hCAR, and “hCAR-T-HL” indicates the result of mice administered with T cells transduced with HL form hCAR. As a result, it was revealed that both LH form and HL form of CAR-T cells effectively inhibited tumor growth compared to hCont-T cells.

Further, FIG. 2F(a) provides a graph showing the results of examining the reactivity of anti-human TIGIT antibody (mouse, clone C18-25) against human TIGIT antigen. This antibody was used to evaluate cytotoxic activity. In FIG. 2F(a), “TIGIT (C18-25)” represents an anti-human TIGIT antibody (mouse, clone C18-25), and “2^{nd} PE” represents a PE-labeled anti-mouse immunoglobulin antibody.

FIG. 2F(b) shows the results of evaluating the cytotoxic activity of GPC1-specific CAR-T cells (hCAR-T-HL) against LK2-hGPC1 when an anti-TIGIT antibody (Anti-TIGIT) was added thereto. As a result, it was revealed that the addition of anti-TIGIT antibody resulted in more effective killing of target cells by hCAR-T cells.

From the above results, it was revealed that the GPC1-specific hCAR-T cells exhibit specific recognition and strong anti-tumor effects against hGPC1-expressing human tumor.

### Experimental Example 3

(Examination on Expression of mGPC1 in Mouse Normal Systemic Tissues)

In Experimental Example 2, all NOG mice transplanted with hCAR-T cells died of severe graft-versus-host disease (GVHD) due to administration of xenogeneic T cells. As such, it is not possible to evaluate on-target/off-tumor toxicity in this model. Therefore, the inventors used syngeneic mouse models for evaluation of on-target/off-tumor toxicity.

First, the mRNA expression level of mGPC1 in various mouse normal tissues was examined by quantitative RT-PCR analysis. GAPDH mRNA was used as an internal control.

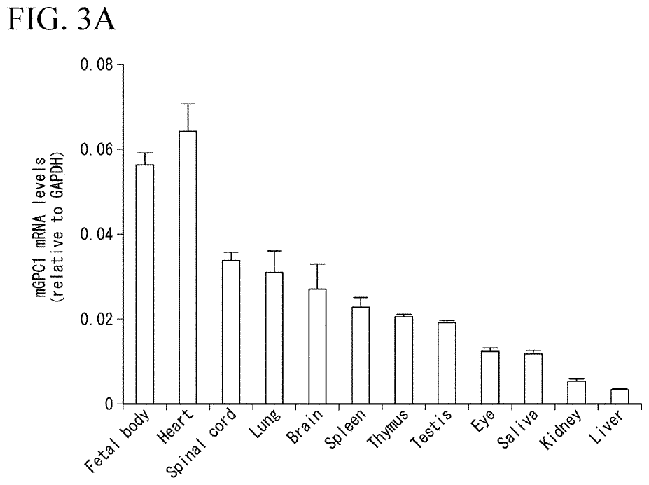

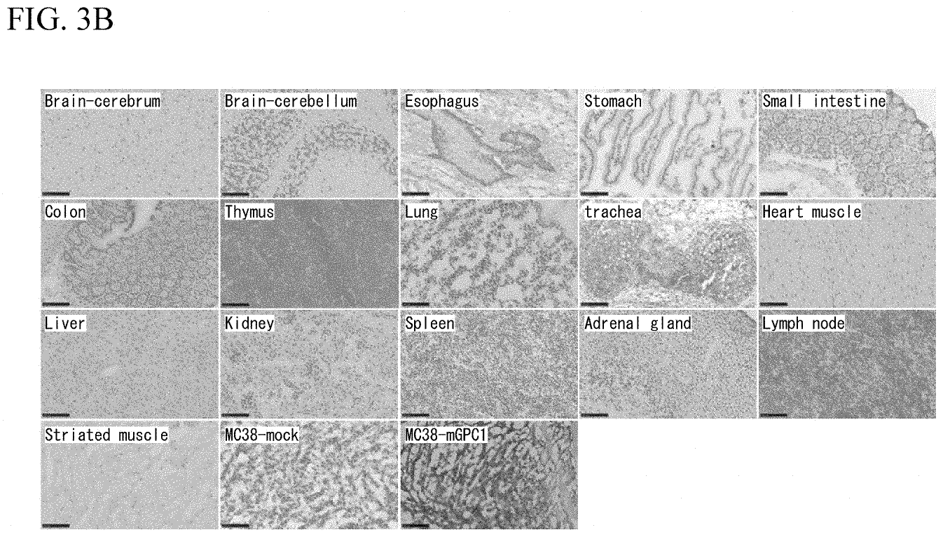

FIG. 3A provides a graph showing the results of quantitative RT-PCR analysis. As a result, it was revealed that a slight, but detectable, expression of mGPC1 mRNA was observed in almost all tissues. Further, from the results of FIGS. 1A and 3A described above, it was revealed that the heart, lung, and brain show relatively high expression of GPC1 mRNA in both human and mouse.

Subsequently, the reactivity of anti-GPC1 monoclonal antibody (clone 1-12) was evaluated against various mouse normal tissues.

FIG. 3B provides representative micrographs of the results of immunostaining. The scale bar indicates 100 μm. The results showed strong and uniform GPC1 expression in MC38-mGPC1 cells (murine colon adenocarcinoma cell line in which mGPC1 is forcibly expressed). On the other hand, it was revealed that normal tissues were not clearly stained by the anti-GPC1 antibody (clone 1-12). There results were similar to the human results in FIG. 1B described above.

The above results indicate that the expression patterns of GPC1 between human and mice at mRNA and protein levels are similar, thereby indicating that syngeneic mouse model (C57BL/6) could be used to evaluate on-target/off-tumor toxicity.

### Experimental Example 4

(Examination of GPC1-Specific mCAR-T Cells)

FIGS. 4A(a) to 4A(d) provide graphs showing the results of examining the reactivity of anti-GPC1 monoclonal antibody (clone 1-12) or isotype control antibody by flow cytometry.

FIG. 4A(a) shows the result of the cell line (LK2-mock) in which the human lung squamous cell carcinoma cell line LK2 is transduced with an empty vector, and FIG. 4A(b) shows the result of the cell line (LK2-hGPC1) in which hGPC1 is forcibly expressed in LK2, FIG. 4A(c) shows the result of the cell line (MC38-mGPC1) in which mGPC1 is forcibly expressed in a murine colon adenocarcinoma cell line (MC38), and FIG. 4A(d) shows the result of the cell line (MC38-mGPC1) in which mGPC1 is forcibly expressed in a murine sarcoma cell line (MCA-205).

As a result, it was revealed that the anti-GPC1 monoclonal antibody (clone 1-12) has reactivity to both hGPC1 and mGPC1. This result indicates that adverse effects can be evaluated using syngeneic mouse models transplanted with mCAR-T cells.

Since the hCAR-T cells (HL form) showed higher anti-tumor activity than the hCAR-T cells (LH form) in Experimental Example 2, mCAR-T cells of the HL form were generated in this Experimental Example.

FIG. 4B provides a schematic diagram showing the structure of mCAR. As shown in FIG. 4B, the human CD3 and CD28 sequences in the hCAR vector (HL form) were converted into homologous mouse sequences to generate an mCAR vector.

Subsequently, activated T cells derived from a transgenic mouse constitutively expressing EGFP were transduced with mCAR vector. Subsequently, in order to evaluate the antigen-specific activity of mCAR-T cells, mCAR-T cells were co-cultured with MC38-mGPC1 cells and subjected to IFNγ secretion assay and ⁵¹Cr release assay.

FIG. 4C provides a graph showing the results of IFNγ secretion assay. In FIG. 4C, “mCont-T” indicates the result of murine T cells not transduced with CAR, “mCAR-T” indicates the result of mCAR-T cells, “non T cell” indicates the result of culturing only tumor cells, “T cells alone” indicates the result of culturing only various T cells for comparison, “MC38-mock” indicates the result of co-culturing various T cells with MC38-mock cells, and “MC38-mGPC1” indicates the result of co-culturing various T cells with MC38-mGPC1 cells. As a result, it was revealed that mCAR-T cells released IFNγ in an mGPC1-specific manner.

FIGS. 4D(a) and 4D(b) provide graphs showing the results of the ⁵¹Cr release assay. In FIGS. 4D(a) and 4D(b), “mCont-T” indicates the result of murine T cells not transduced with CAR, and “mCAR-T” indicates the result of mCAR-T cells. In FIGS. 4D(a) and 4D(b), the vertical axis represents the specific lysis of cells measured by the release of ⁵¹Cr, and the horizontal axis represents the mixing ratio of T cells and cancer cells (T cells: cancer cells).

FIG. 4D(a) shows the results of co-culturing various T cells with MC38-mock cells, and FIG. 4D(b) shows the results of co-culturing various T cells with MC38-mGPC1 cells. As a result, it was revealed that mCAR-T cells killed target cells in an mGPC1-specific manner.

Subsequently, the inventors intravenously administered the mCAR-T cells to syngeneic mouse models transplanted with MC38-mGPC1 cells or MCA205-mGPC1 cells, and examined the anti-tumor activity of CAR-T cells in vivo. The mCAR-T cells or murine T cells not transduced with CAR were intravenously administered at a dose of 2×10⁶ cells/mouse on day 3 after the transplantation of cancer cells, and the time course of tumor volume was measured.

FIGS. 4E(a) to 4E(c) and FIGS. 4F(a) to 4F(c) provide graphs showing the measurement results of tumor volume. FIGS. 4E(a) to 4E(c) show the results of mice transplanted with MC38-mGPC1 cells. FIG. 4E(a) provides a graph showing the mean value of the results of the mice in the respective groups, FIG. 4E(b) provides a graph showing the individual results of mice transplanted with murine T cells not transduced with CAR (mCont-T), and FIG. 4E(c) provides a graph showing the individual results of mice transplanted with mCAR-T cells.

As a result, it was revealed that mCAR-T cells effectively suppressed tumor growth in the MC38-mGPC1 mouse model, as compared with mCont-T cells.

Further, FIGS. 4F(a) to 4F(c) show the results of mice transplanted with MCA205-mGPC1 cells. FIG. 4F(a) provides a graph showing the mean value of the results of the mice in the respective groups, FIG. 4F(b) provides a graph showing the individual results of mice transplanted with murine T cells not transduced with CAR (mCont-T), and FIG. 4F(c) provides a graph showing the individual results of mice transplanted with mCAR-T cells.

As a result, in the MCA205-mGPC1 mouse model, complete eradication of the tumor was observed in 4 out of 5 mice and was maintained for at least 100 days.

From the above results, it was revealed that mCAR-T cells effectively suppressed tumor growth as compared with mCont-T cells.

Subsequently, the in vivo persistence of mCAR-T cells was evaluated using the MC38-mGPC1 mouse model. Specifically, the ratio of GFP-positive CD8-positive mCAR-T cells in total CD8-positive T cells derived from peripheral blood and tumor tissue on day 15 from the start of the experiment was measured.

FIGS. 4G(a) and 4G(b) provide graphs showing the measurement results of the ratio of GFP-positive CD8-positive mCAR-T cells in total CD8-positive T cells derived from peripheral blood and tumor tissue, respectively. The graphs show a plot for each mouse. As a result, it was revealed that mCAR-T cells efficiently persisted in peripheral blood and infiltrated into the tumor in MC38-mGPC1 tumor-bearing mice.

Further, in the MCA205-mGPC1 mouse model, GFP-positive CD8-positive mCAR-T cells were detected in peripheral blood of mice showing complete tumor eradication on day 60 from the start of the experiment, confirming long-term mCAR-T cell survival (FIG. 4G(c)).

Subsequently, the functional persistence of mCAR-T cells was evaluated by assessing IFNγ secretion. On day 15 from the start of the experiment, total CD8-positive T cells including transplanted mCAR-T cells were harvested from the spleen and tumor-infiltrating lymphocytes of MC38-mGPC1 mouse model. Subsequently, the cells were co-cultured with LK2-hGPC1 cells or LK2-mock cells as stimulator cells, and the concentration of mouse IFNγ in the supernatant was measured.

FIGS. 4H(a) and 4H(b) provide graphs showing the measurement results of IFNγ concentration. FIG. 4H(a) shows the result of cells derived from spleen, and FIG. 4H(b) shows the result of tumor-infiltrating lymphocytes (TIL). In FIGS. 4H(a) and 4H(b), “Non stimulator” indicates the result of not co-culturing with stimulator cells, “LK2-mock” indicates the result of co-culturing with LK2-mock cells, “LK2-hGPC1” indicates the result of co-culturing with LK2-hGPC1 cells, “mCont-T” indicates the result of murine T cells not transduced with CAR, and “rnCAR-T” indicates the result of mCAR-T cells.

As a result, it was revealed that the mCAR-T cells were functional even on day 15 from the start of the experiment.

Next, the inventors examined whether CAR-T cells targeting a single antigen could enhance the induction of T cell responses against other endogenous antigens. In order to evaluate the antigen-specific T cell response, the induction of gp70-specific T cells was evaluated through IFNγ release assay in which the CD8-positive tumor-infiltrating lymphocytes were restimulated with gp70 peptide (SEQ ID NO: 7) as a stimulator or f3-gal peptide (SEQ ID NO: 8) as a control.

FIG. 4I provides a graph showing the measurement results of IFNγ concentration in the supernatant. In FIG. 4I, “mCont-T” indicates the result of mice administered with the murine T cells not transduced with CAR, and “mCAR-T” indicates the result of mice administered with the mCAR-T cells.

As a result, it was revealed that the induction of gp70-specific T cells in CD8-positive tumor-infiltrating lymphocytes was significantly enhanced by the administration of mCAR-T cells.

From the above results, it was revealed that mCAR-T cells can functionally persist in vivo, eliminate solid tumors established without obvious side effects, and enhance endogenous anti-tumor response against tumor antigens other than GPC1.

### Experimental Example 5

(Examination of Side Effects Caused by GPC1-Specific mCAR-T Cells)

In order to evaluate the adverse effects of mCAR-T cells in vivo, clinical symptoms and histological abnormalities were analyzed.

First, mCAR-T cells or murine T cells not transduced with CAR were intravenously administered to syngeneic mouse model transplanted with MC38-mGPC1 cells or MCA205-mGPC1 cells on day 3 from the transplantation of cancer cells.

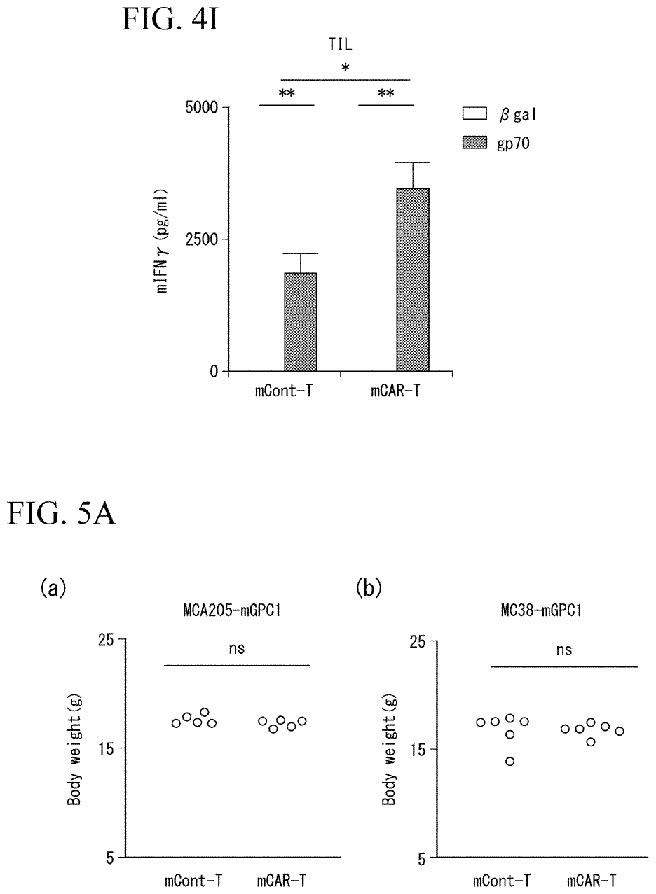

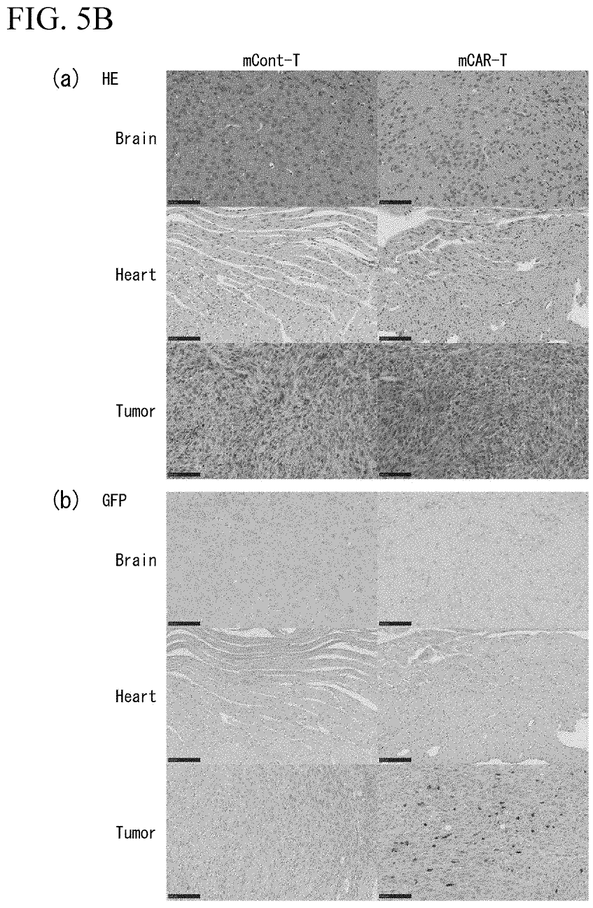

FIGS. 5A(a) and 5A(b) provide graphs showing the results of measuring body weight in syngeneic mouse models on day 15 from the transplantation of cancer cells. FIG. 5A(a) shows the result of the syngeneic mouse model transplanted with MC38-mGPC1 cells, and FIG. 5A(b) shows the result of the syngeneic mouse model transplanted with MCA205-mGPC1 cells. In FIGS. 5A(a) and 5A(b), “mCont-T” indicates the result of mice transplanted with murine T cells not transduced with CAR, “mCAR-T” indicates the result of mice transplanted with mCAR-T cells, and “ns” indicates that there was no significant difference.

As a result, it was revealed that, in any of the syngeneic mouse models, the body weight of mice to which the mCAR-T cells were administered did not change significantly compared to the body weight of mice to which the mCont-T cells were administered. Further, in any of the syngeneic mouse models, the appearance and behavior of the mice to which the mCAR-T cells were administered were not different from the appearance and behavior of the mice to which the mCont-T cells were administered.

Subsequently, for each syngeneic mouse model, normal tissues and tumor tissues were subjected to hematoxylin/eosin staining and immunostaining with an anti-GFP antibody. FIG. 5B(a) provides micrographs showing the results of hematoxylin/eosin staining. The scale bar is 100 μm. FIG. 5B(b) provides micrographs showing the results of immunostaining. The scale bar is 100 μm.

In FIGS. 5B(a) and 5B(b), “mCont-T” indicates the result of mice transplanted with murine T cells not transduced with CAR, “mCAR-T” indicates the result of mice transplanted with mCAR-T cells, “HE” indicates the result of hematoxylin/eosin staining, and “GFP” indicates the result of immunostaining using an anti-GFP antibody.

The results showed no obvious histological tissue damage or infiltration of administered mCAR-T cells in any of the tested normal tissues including the heart and brain where GPC1 mRNA expression was detected by quantitative RT-PCR in Experimental Example 1 and Experimental Example 3.

Further, normal systemic tissues were also subjected to hematoxylin/eosin staining and immunostaining using an anti-GFP antibody. The results showed no obvious histological tissue damage in any of the normal systemic tissues. Incidentally, slight infiltration of the administered mCAR-T cells was observed in normal systemic tissues.

From the above results, it was revealed that mCAR-T cells can eliminate mGPC1-positive tumors without obvious adverse effects, despite slight GPC1 mRNA expression in some of the normal tissues.

### Experimental Example 6

(GPC1-Specific mCAR-T Cell Therapy Combined with Anti-PD-1 Antibody Therapy)

First, mCAR-T cells or murine T cells not transduced with CAR were intravenously administered to syngeneic mouse model transplanted with MC38-mGPC1 cells on day 3 from the transplantation of cancer cells. Subsequently, the expression of PD-1 on the endogenous CD8-positive T cells and the administered CD8-positive T cells in the tumor tissues of mice on day 15 from the transplantation of cancer cells was examined.

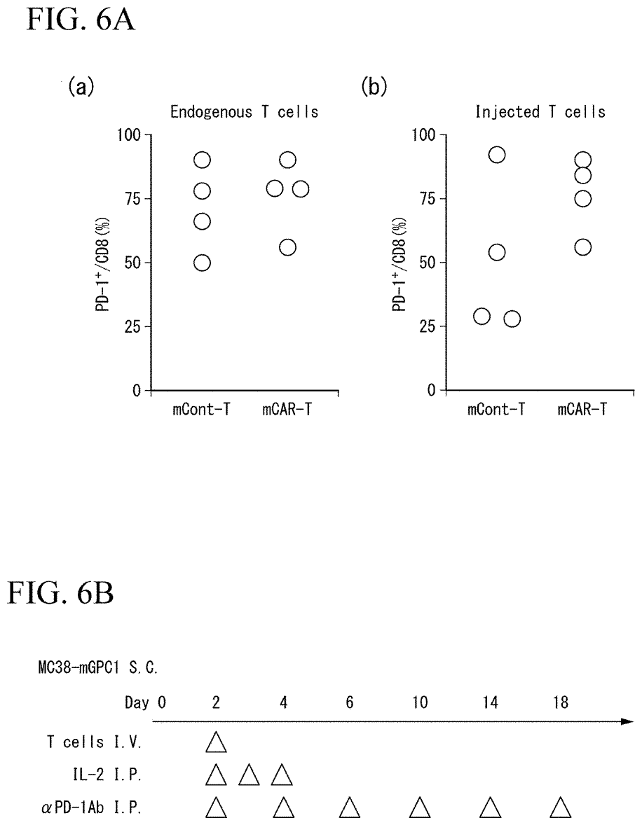

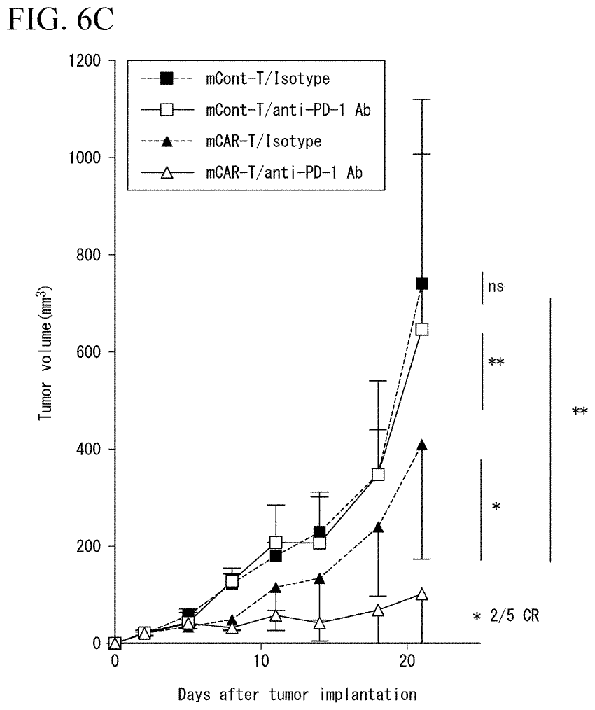

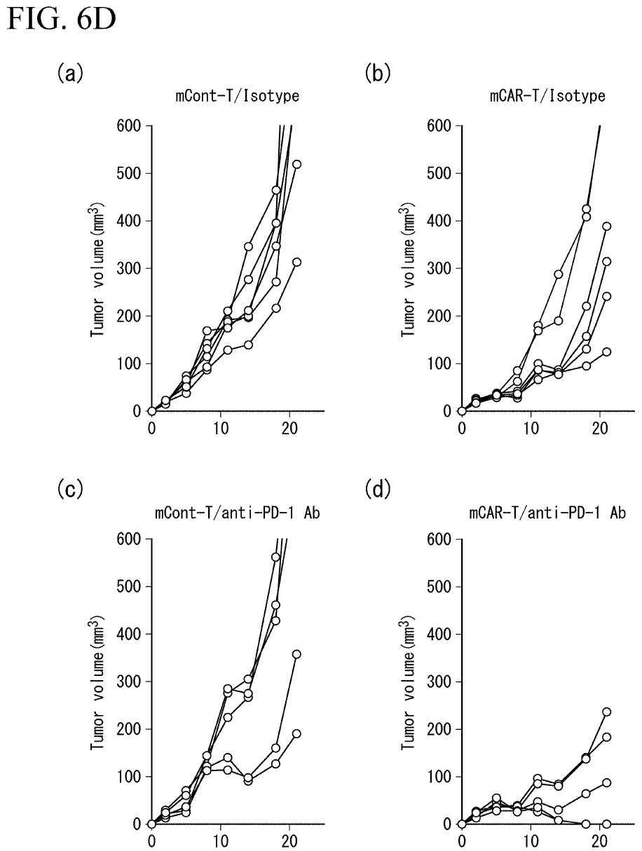

FIG. 6A(a) provides a graph showing the results of endogenous CD8-positive T cells, and FIG. 6A(b) provides a graph showing the results of administered CD8-positive T cells. In FIGS. 6A(a) and 6A(b), “mCont-T” indicates the result of mice transplanted with murine T cells not transduced with CAR, and “mCAR-T” indicates the result of mice transplanted with mCAR-T cells.

As a result, it was revealed that most mCAR-T cells and endogenous T cells present in the tumor tissue express PD-1 in the MC38-mGPC1 mouse model. Thus, it was examined whether administration of anti-PD-1 antibody enhances the anti-tumor activity by the administration of mCAR-T cells.

FIG. 6B provides a diagram showing the experimental schedule. On day 0, mice were injected subcutaneously with MC38-mGPC1 cells. Subsequently, on day 2, 2×10⁶ cells/mouse of T cells was intravenously administered. Further, IL-2 was intraperitoneally administered on days 2, 3, and 4. Further, 200 mg/mouse of anti-PD-1 antibody was intraperitoneally administered on days 2, 4, 6, 10, 14, and 18. In addition, the tumor volume was measured with time after the transplantation of MC38-mGPC1 cells.

FIG. 6C provides a graph showing the measurement results of tumor volume of mice in the respective groups. In FIG. 6C, “mCont-T” indicates the result of mice administered with murine T cells not transduced with CAR, “Isotype” indicates the result of mice administered with the isotype control antibody, “Anti-PD-1Ab” indicates the result of mice administered with anti-PD-1 antibody, “mCAR-T” indicates the result of mice administered with mCAR-T cells, and “CR” indicates complete response.

Further, FIGS. 6D(a) to 6D(d) provide graphs showing the measurement results of tumor volume of mice in the respective groups for each individual mouse. FIG. 6D(a) shows the results of mice administered with murine T cells not transduced with CAR and isotype control antibody, and FIG. 6D(b) shows the results of mice administered with mCAR-T cells and isotype control antibody, FIG. 6D(c) shows the results of mice administered with murine T cells not transduced with CAR and anti-PD-1 antibody, and FIG. 6D(d) shows the results of mice administered with mCAR-T cells and anti-PD-1 antibody.

As a result, it was revealed that combining mCAR-T cell therapy with an immune checkpoint inhibitor, exemplified by the anti-PD-1 antibody, maintained the cytotoxic activity of mCAR-T cells after day 8. On the other hand, mCAR-T cell therapy alone had a significant but weak anti-tumor effect. Taken together with the fact that the immune checkpoint inhibitor (anti-PD-1 antibody) alone could not exhibit the anti-tumor effect, it has been indicated that adjunctive use of immune checkpoint inhibitors such as anti-PD-1 antibody concomitantly with mCAR-T cell therapy can maintain the anti-tumor effect originally possessed by mCAR-T cells for a long period of time and maximize the medicinal effect. Further, since the mCAR-T cells and the immune checkpoint inhibitor were administered on the same day as shown in the administration schedule shown in FIG. 6B, a complex of immune checkpoint inhibitor, such as anti-PD-1 antibody, bound to the surface of mCAR-T cells may also exert medicinal effects. Furthermore, all mice survived without apparent weakness findings, and all mice showed no adverse effects from clinical symptoms.

The above results indicate that the anti-tumor effect of GPC1-specific CAR-T cell therapy is maintained by combining the anti-PD-1 monoclonal antibody. The results also indicate that there are no visible side effects.

### INDUSTRIAL APPLICABILITY

According to the present invention, it is possible to provide a medicament composition, a kit, and a technique for effectively treating GPC1-positive cancer.

1. A medicament for cancer treatment, comprising as an active ingredient T cells having a chimeric antigen receptor that binds to glypican 1 (GPC1), wherein the medicament is administered concomitantly with an immune checkpoint inhibitor according to regimens (a) and (b) to maintain anti-tumor activity of the T cells:
(a) administering an effective amount of the T cells to a cancer patient; and
(b) continuously administering 0.01 mg/kg body weight to 100 mg/kg body weight of the immune checkpoint inhibitor per dose to the cancer patient every 1 to 5 weeks.

2. The medicament for cancer treatment according to claim 1, wherein the immune checkpoint inhibitor is at least one selected from the group consisting of an anti-PD-1 antibody, an anti-PD-L1 antibody, an anti-CTLA-4 antibody, an anti-TIGIT antibody, an anti-CD80 (B7-1) antibody, an anti-LAG-3 antibody, and an anti-TIM3 antibody.

3. The medicament for cancer treatment according to claim 2, wherein the immune checkpoint inhibitor is an anti-PD-1 antibody or an anti-TIGIT antibody.

4. The medicament for cancer treatment according to claim 3, wherein the immune checkpoint inhibitor is an anti-PD-1 antibody.

5. The medicament for cancer treatment according to claim 4, wherein the anti-PD-1 antibody is nivolumab.

6. The medicament for cancer treatment according to claim 1, wherein the chimeric antigen receptor contains a GPC1 binding domain, a transmembrane domain, a costimulatory domain, and a cytoplasmic signal domain.

7. The medicament for cancer treatment according to claim 6, wherein the GPC1 binding domain includes a heavy chain variable region including a heavy chain CDR1 consisting of an amino acid sequence of SEQ ID NO: 9, a heavy chain CDR2 consisting of an amino acid sequence of SEQ ID NO: 10, and a heavy chain CDR3 consisting of an amino acid sequence of SEQ ID NO: 11, and a light chain variable region including a light chain CDR1 consisting of an amino acid sequence of SEQ ID NO: 12, a light chain CDR2 consisting of an amino acid sequence of SEQ ID NO: 13, and a light chain CDR3 consisting of an amino acid sequence of SEQ ID NO: 14.

8. The medicament for cancer treatment according to claim 6, wherein the GPC1 binding domain consists of a protein consisting of an amino acid sequence of SEQ ID NO: 15, or consists of a protein consisting of an amino acid sequence having a sequence identity of 95% or more with the amino acid sequence of SEQ ID NO: 15 and binds to GPC1.

9. The medicament for cancer treatment according to claim 6, wherein the GPC1 binding domain is humanized.

10. The medicament for cancer treatment according to claim 1, wherein the cancer is a cancer selected from the group consisting of esophageal cancer, cervical cancer, breast cancer, pancreatic cancer, glioma, mesothelioma, thyroid cancer, lung cancer, liver cancer, colon cancer, head and neck cancer, urothelial cancer, ovarian cancer, melanoma, and prostate cancer.

11. The medicament for cancer treatment according to claim 10, wherein the cancer is esophageal cancer.

12. An anti-tumor activity maintaining agent for T cells having a chimeric antigen receptor that binds to GPC1, the agent comprising as an active ingredient an immune checkpoint inhibitor, wherein the agent is used by administering concomitantly with the T cells according to regimens (a) and (b):
(a) administering an effective amount of the T cells to a cancer patient; and
(b) continuously administering 0.01 mg/kg body weight to 100 mg/kg body weight of the immune checkpoint inhibitor per dose to the cancer patient every 1 to 5 weeks.

13. (canceled)

14. A medicament for cancer treatment, comprising as an active ingredient a complex of a T cell having a chimeric antigen receptor that binds to GPC1 and an immune checkpoint inhibitor.

15. The medicament for cancer treatment according to claim 14, wherein the immune checkpoint inhibitor is at least one selected from the group consisting of an anti-PD-1 antibody, an anti-PD-L1 antibody, an anti-CTLA-4 antibody, an anti-TIGIT antibody, an anti-CD80 (B7-1) antibody, an anti-LAG-3 antibody, and an anti-TIM3 antibody.

16. The medicament for cancer treatment according to claim 15, wherein the immune checkpoint inhibitor is an anti-PD-1 antibody or an anti-TIGIT antibody.

17. The medicament for cancer treatment according to claim 16, wherein the immune checkpoint inhibitor is an anti-PD-1 antibody.

18. The medicament for cancer treatment according to claim 17, wherein the anti-PD-1 antibody is nivolumab.

19-22. (canceled)


SEQUENCE LISTING (16 sequences)

SEQ ID NO: 1 - protein, 123 aa, Gallus gallus
AVTLDESGGGLQTPGRALSLVCKASGFTFSRYAMYWVRQAPGKGLEFVAGIGNTGRYTGYGSAVKGRATISRDSGQSTVRLQLNNLRAEDTGNYYCAKSVSPYCCDAADIDAWGHGTEVIVSS

SEQ ID NO: 2 - protein, 106 aa, Gallus gallus
ASTQPSSVSANPGETVKITCSGGSSGYAYGWYQQKSPGSAPVTLLYSNNNRPSDIPSRFSGSKSGSTATLTITGVQAEDEAVYFCGSVDSSSYAGIFGAGTTLTVL

SEQ ID NO: 3 - protein, 15 aa, Artificial Sequence
  Synthesized peptide linker
GGGGSGGGGSGGGGS

SEQ ID NO: 4 - DNA, 1464 bp, Artificial Sequence
  Nucleic acid sequence derived from Gallus gallus and Homo Sapiens
atggccttaccagtgaccgccttgctcctgccgctggccttgctgctccacgccgccaggccggctagcactcagccgtcctcggtgtcagcaaacccaggagaaaccgtcaagatcacctgctccgggggtagcagtggctatgcttatggctggtaccagcagaagtctcctggcagtgcccctgtcactctgctctatagcaacaacaacagaccctcggacatcccttcacgattctccggttccaaatccggctccacagccacattaaccatcactggggtccaagccgaggacgaggctgtctatttctgtgggagtgtagacagcagcagttatgctggtatatttggggccgggacaaccctgaccgtcctaggcggtggcggatcaggtggcggtggaagtggcggtggtgggtctgcggtgacgttggacgagtccgggggcggcctccagacgcccggaagagcgctcagcctcgtctgtaaggcctccgggttcaccttcagccgttacgccatgtactgggtgcgacaggcgcccggcaaggggctggagttcgtcgctggtattggcaacactggtagatacacaggctacgggtcggcggtgaagggccgtgccaccatctcgagggacagcgggcagagcacagtgaggctgcaactgaacaacctcagggctgaggacaccggcaactactactgcgccaaaagtgttagtccttactgttgtgatgctgctgacatcgacgcatggggccacgggaccgaagtcatcgtctcctccgcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 5 - DNA, 1464 bp, Artificial Sequence
  Nucleic acid sequence derived from Gallus gallus and Homo Sapiens
atggccttaccagtgaccgccttgctcctgccgctggccttgctgctccacgccgccaggccggcggtgacgttggacgagtccgggggcggcctccagacgcccggaagagcgctcagcctcgtctgtaaggcctccgggttcaccttcagccgttacgccatgtactgggtgcgacaggcgcccggcaaggggctggagttcgtcgctggtattggcaacactggtagatacacaggctacgggtcggcggtgaagggccgtgccaccatctcgagggacagcgggcagagcacagtgaggctgcaactgaacaacctcagggctgaggacaccggcaactactactgcgccaaaagtgttagtccttactgttgtgatgctgctgacatcgacgcatggggccacgggaccgaagtcatcgtctcctccggcggtggcggatcaggtggcggtggaagtggcggtggtgggtctgctagcactcagccgtcctcggtgtcagcaaacccaggagaaaccgtcaagatcacctgctccgggggtagcagtggctatgcttatggctggtaccagcagaagtctcctggcagtgcccctgtcactctgctctatagcaacaacaacagaccctcggacatcccttcacgattctccggttccaaatccggctccacagccacattaaccatcactggggtccaagccgaggacgaggctgtctatttctgtgggagtgtagacagcagcagttatgctggtatatttggggccgggacaaccctgaccgtcctagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 6 - DNA, 1476 bp, Artificial Sequence
  Nucleic acid sequence derived from Gallus gallus and Mus musculus
atggcctcaccgttgacccgctttctgtcgctgaacctgctgctgctgggtgagtcgattatcctggggagtggagaagctgcggtgacgttggacgagtccgggggcggcctccagacgcccggaagagcgctcagcctcgtctgtaaggcctccgggttcaccttcagccgttacgccatgtactgggtgcgacaggcgcccggcaaggggctggagttcgtcgctggtattggcaacactggtagatacacaggctacgggtcggcggtgaagggccgtgccaccatctcgagggacagcgggcagagcacagtgaggctgcaactgaacaacctcagggctgaggacaccggcaactactactgcgccaaaagtgttagtccttactgttgtgatgctgctgacatcgacgcatggggccacgggaccgaagtcatcgtctcctccggcggtggcggatcaggtggcggtggaagtggcggtggtgggtctgctagcactcagccgtcctcggtgtcagcaaacccaggagaaaccgtcaagatcacctgctccgggggtagcagtggctatgcttatggctggtaccagcagaagtctcctggcagtgcccctgtcactctgctctatagcaacaacaacagaccctcggacatcccttcacgattctccggttccaaatccggctccacagccacattaaccatcactggggtccaagccgaggacgaggctgtctatttctgtgggagtgtagacagcagcagttatgctggtatatttggggccgggacaaccctgaccgtcctagcggccgcaattgagttcatgtaccctccgccttacctagacaacgagaggagcaatggaactattattcacataaaagagaaacatctttgtcatactcagtcatctcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 7 - protein, 8 aa, Murine leukemia virus
KSPWFTTL

SEQ ID NO: 8 - protein, 8 aa, Escherichia coli
DAPIYTNV

SEQ ID NO: 9 - protein, 5 aa, Gallus gallus
RYAMY

SEQ ID NO: 10 - protein, 17 aa, Gallus gallus
GIGNTGRYTGYGSAVKG

SEQ ID NO: 11 - protein, 14 aa, Gallus gallus
SVSPYCCDAADIDA

SEQ ID NO: 12 - protein, 10 aa, Gallus gallus
SGGSSGYAYG

SEQ ID NO: 13 - protein, 7 aa, Gallus gallus
SNNNRPS

SEQ ID NO: 14 - protein, 11 aa, Gallus gallus
GSVDSSSYAGI

SEQ ID NO: 15 - protein, 487 aa, Artificial Sequence
  Amino acid sequence derived from Gallus gallus and Homo Sapiens
MALPVTALLLPLALLLHAARPAVTLDESGGGLQTPGRALSLVCKASGFTFSRYAMYWVRQAPGKGLEFVAGIGNTGRYTGYGSAVKGRATISRDSGQSTVRLQLNNLRAEDTGNYYCAKSVSPYCCDAADIDAWGHGTEVIVSSGGGGSGGGGSGGGGSASTQPSSVSANPGETVKITCSGGSSGYAYGWYQQKSPGSAPVTLLYSNNNRPSDIPSRFSGSKSGSTATLTITGVQAEDEAVYFCGSVDSSSYAGIFGAGTTLTVLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 244 aa, Artificial Sequence
  Amino acid sequence derived from Gallus gallus
AVTLDESGGGLQTPGRALSLVCKASGFTFSRYAMYWVRQAPGKGLEFVAGIGNTGRYTGYGSAVKGRATISRDSGQSTVRLQLNNLRAEDTGNYYCAKSVSPYCCDAADIDAWGHGTEVIVSSGGGGSGGGGSGGGGSASTQPSSVSANPGETVKITCSGGSSGYAYGWYQQKSPGSAPVTLLYSNNNRPSDIPSRFSGSKSGSTATLTITGVQAEDEAVYFCGSVDSSSYAGIFGAGTTLTVL